(19) United States
(12) Patent Application Publication
(10) Pub. No.: US 2015/0328199 A1
(43) Pub. Date: Nov. 19, 2015

(54) METHODS AND COMPOSITIONS USING 4-AMINO-2-(2,6-DIOXO-PIPERIDINE-3-YL)-ISOINDOLINE-1,3-DIONE FOR TREATMENT OF CANCERS

(71) Applicant: Celgene Corporation, Summit, NJ (US)

(72) Inventors: Yan LI, Warren, NJ (US); Maria PALMISANO, North Chatham, NH (US); Simon ZHOU, Malvern, PA (US); Nianhang CHEN, Basking Ridge, NJ (US); Angela J. JAMES, Morris Plains, NJ (US)

(21) Appl. No.: 14/713,655

(22) Filed: May 15, 2015

Related U.S. Application Data
(60) Provisional application No. 61/994,766, filed on May 16, 2014.

(51) Int. Cl. A61K 31/454; A61K 45/06

(52) U.S. Cl. CPC A61K 31/454; A61K 45/06

(57) ABSTRACT

Methods and compositions for treating, preventing or managing cancers are disclosed. The methods encompass the administration of 4-amino-2-(2,6-dioxo-piperidine-3-yl)-isoindoline-1,3-dione, also known as Pomalidomide. Furthermore, provided herein are methods of treatment using this compound with chemotherapy, radiation therapy, hormonal therapy, biological therapy or immunotherapy. Pharmaceutical compositions and single unit dosage forms suitable for use in the methods provided herein are also disclosed.

### 1. CROSS REFERENCE TO RELATED APPLICATIONS

This application claims the benefit of the priority of U.S. Provisional Application No. 61/994,766, filed May 16, 2014, the disclosure of which is incorporated herein by reference in its entirety.

### 2. FIELD

Provided herein are methods of treating, preventing and/or managing cancers, which comprise administering to a patient 4-amino-2-(2,6-dioxo-piperidine-3-yl)-isoindoline-1,3-dione, or an enantiomer or a mixture of enantiomers thereof, or a pharmaceutically acceptable salt, solvate, hydrate, co-crystal, clathrate, or polymorph thereof.

### 3. BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3rd ed., Mosby, St. Louis, Mo., 1993).

Solid tumors are abnormal masses of tissue that may, but usually do not contain cysts or liquid areas. Solid tumors may be benign (not cancer), or malignant (cancer). Different types of solid tumors are named for the type of cells that form them. Examples of solid tumors include, but are not limited to malignant melanoma, adrenal carcinoma, breast carcinoma, renal cell cancer, carcinoma of the pancreas, non-small-cell lung carcinoma (NSCLC) and carcinoma of unknown primary. Drugs commonly administered to patients with various types or stages of solid tumors include, but are not limited to, celebrex, etoposide, cyclophosphamide, docetaxel, apecitabine, IFN, tamoxifen, IL-2, GM-CSF, or a combination thereof.

The treatment of solid tumors has encountered several severe drawbacks in clinical practice because chemotherapeutic agents can also affect healthy cells of the body, resulting in severe side effects such as nausea, vomiting, immunotoxicity, cardiotoxicty, liver damage, etc. There have been attempts to deliver anti-tumor agent via direct injection or by targeting tumor-specific surface antigens, all with mixed results in animal and human trials. These approaches require complex and sometimes anatomically unfeasible procedures or sophisticated understanding of tumor biology to identify tumor specific targets while sparing healthy tissues.

In order to circumvent the above disadvantages, there is a need to develop chemotherapeutic agents that target tumor cells, thereby minimizing the burden on healthy tissues and organs. There also exists a need for chemotherapeutic agents with improved pharmacokinetic parameters and efficacy.

### 4. SUMMARY

In certain embodiments, provided herein are methods of treating and preventing cancer, including primary and metastatic cancer, as well as cancer that is refractory or resistant to conventional chemotherapy, which comprise administering to a patient in need of such treatment or prevention a therapeutically or prophylactically effective amount of 4-amino-2-(2,6-dioxo-piperidine-3-yl)-isoindoline-1,3-dione, having the structure of Formula I:

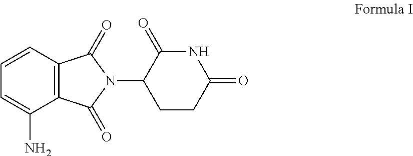 [SMILES: Nc1cccc2c1C(=O)N(C1CCC(=O)NC1=O)C2=O]

or an enantiomer or a mixture of enantiomers thereof, or a pharmaceutically acceptable salt, solvate, hydrate, co-crystal, clathrate, or polymorph thereof as a single agent or as a part of a combination therapy. In one embodiment, the cancer is solid tumor. In one embodiment, the solid tumor is selected from the group consisting of melanoma, breast cancer, head and neck tumors, breast carcinoma, non-small cell lung carcinoma, ovarian carcinoma, pancreatic carcinoma, prostate carcinoma, colorectal carcinoma, and hepatocellular carcinoma. In one embodiment, the solid tumor is lenalidomide resistant tumor.

In one embodiment, provided herein is a method for treating, preventing or managing cancer while minimizing adverse side effects in a patient, wherein the method comprises administering to the patient a therapeutically or prophylactically effective amount 4-amino-2-(2,6-dioxo-piperidine-3-yl)-isoindoline-1,3-dione, or an enantiomer or a mixture of enantiomers thereof, or a pharmaceutically acceptable salt, solvate, hydrate, co-crystal, clathrate, or polymorph thereof.

In one embodiment, provided herein is a method for selectively targeting cancer cells in a patient comprising administering to the patient a therapeutically or prophylactically effective amount 4-amino-2-(2,6-dioxo-piperidine-3-yl)-isoindoline-1,3-dione, or an enantiomer or a mixture of enantiomers thereof, or a pharmaceutically acceptable salt, solvate, hydrate, co-crystal, clathrate, or polymorph thereof.

Further provided herein are methods of treating, preventing, or managing cancer, comprising administering to a patient in need of such treatment, prevention, or management a therapeutically or prophylactically effective amount of 4-amino-2-(2,6-dioxo-piperidine-3-yl)-isoindoline-1,3-dione, or an enantiomer or a mixture of enantiomers thereof, or a pharmaceutically acceptable salt, solvate, hydrate, co-crystal, clathrate, or polymorph thereof; in combination with a therapy conventionally used to treat, prevent, or manage cancer. Examples of such conventional therapies include, but are not limited to, surgery, chemotherapy, radiation therapy, hormonal therapy, biological therapy, and immunotherapy.

In certain embodiments, provided herein are pharmaceutical compositions, single unit dosage forms, and dosing regimens which comprise 4-amino-2-(2,6-dioxo-piperidine-3-yl)-isoindoline-1,3-dione, or a pharmaceutically acceptable salt, solvate (e.g., hydrate), stereoisomer, clathrate, or prodrug thereof, and a second, or additional, active agent or ingredient. Second active agents or ingredients include specific combinations, or “cocktails,” of drugs or therapy, or both. In one embodiment, the pharmaceutical composition provided herein comprises 4-amino-2-(2,6-dioxo-piperidine-3-yl)-isoindoline-1,3-dione, or a pharmaceutically acceptable salt, solvate (e.g., hydrate), stereoisomer, clathrate, or prodrug thereof, wherein the composition is for oral administration.

### 5. BRIEF DESCRIPTION OF FIGURES

FIG. 1 shows distribution of PK samples per subject in the population pharmacokinetic dataset for 4-amino-2-(2,6-dioxo-piperidine-3-yl)-isoindoline-1,3-dione.

FIG. 2 shows individual dose normalized 4-amino-2-(2,6-dioxo-piperidine-3-yl)-isoindoline-1,3-dione concentration versus time profiles for healthy normal subjects versus multiple myeloma (MM) patients. The longer terminal phase in MM patients than in healthy normal subjects indicates possibly deeper tissue/organ distribution of 4-amino-2-(2,6-dioxo-piperidine-3-yl)-isoindoline-1,3-dione in MM patients.

FIG. 3A shows concentration vs. time profiles for 4-amino-2-(2,6-dioxo-piperidine-3-yl)-isoindoline-1,3-dione and 2-(2,6-dioxopiperidin-3-yl)-1H-isoindole-1,3(2H)-dione.

FIG. 3B shows concentration vs. time profiles for 4-amino-2-(2,6-dioxo-piperidine-3-yl)-isoindoline-1,3-dione and 3-(4-amino-l-oxo-1,3-dihydro-2H-isoindol-2-yl)piperidine-2,6-dione.

FIG. 4 shows concentration vs. time profiles for 2-(2,6-dioxopiperidin-3-y1)-1H-isoindole-1,3(2H)-dione in patient vs. healthy subjects.

FIG. 5 shows concentration vs. time profiles for 3-(4-amino-l-oxo-1,3-dihydro-2H-isoindol-2-yl)piperidine-2,6-dione in patient vs. healthy subjects.

FIG. 6 shows concentration vs. time profiles for 4-amino-2-(2,6-dioxo-piperidine-3-yl)-isoindoline-1,3-dione in patient vs. healthy subjects.

FIG. 7A shows drug exposure vs. time profiles for 2-(2,6-dioxopiperidin-3-yl)-1H-isoindole-1,3(2H)-dione in patient vs. healthy subjects in plasma compartment.

FIG. 7B shows drug exposure vs. time profiles for 2-(2,6-dioxopiperidin-3-yl)-1H-isoindole-1,3(2H)-dione in patient vs. healthy subjects in peripheral compartment. The drug exposures in central and peripheral compartments were comparable in healthy subjects and patients

FIG. 8A shows drug exposure vs. time profiles for 4-amino-2-(2,6-dioxo-piperidine-3-yl)-isoindoline-1,3-dione in patient vs. healthy subjects in plasma compartment.

FIG. 8B shows drug exposure vs. time profiles for 4-amino-2-(2,6-dioxo-piperidine-3-yl)-isoindoline-1,3-dione in patient vs. healthy subjects in peripheral compartment. The drug exposures in central compartment was comparable in healthy subjects and patients, however higher drug exposure in peripheral compartments in patients as compared to healthy subjects was observed.

FIG. 9 shows that higher disease stage was correlated with higher peripheral compartment 4-amino-2-(2,6-dioxo-piperidine-3-yl)-isoindoline-1,3-dione exposure in patients.

### 6. DETAILED DESCRIPTION

In certain embodiments, provided herein are methods of selectively targeting cancer cells, while leaving healthy cells intact, in a patient comprising administering to the patient in need thereof a therapeutically effective amount of a compound, wherein the compound is 4-amino-2-(2,6-dioxo-piperidine-3-yl)-isoindoline-1,3-dione, an enantiomer, a mixture of enantiomers, a pharmaceutically acceptable salt, solvate, hydrate, co-crystal, clathrate, or polymorph thereof as a single agent or as a part of a combination therapy.

In certain embodiments, provided herein are methods of enhancing selectivity against cancer cells as compared to healthy cells in a patient comprising administering to the patient in need thereof a therapeutically effective amount of a compound, wherein the compound is 4-amino-2-(2,6-dioxo-piperidine-3-yl)-isoindoline-1,3-dione, an enantiomer, a mixture of enantiomers, a pharmaceutically acceptable salt, solvate, hydrate, co-crystal, clathrate, or polymorph thereof as a single agent or as a part of a combination therapy, wherein selectivity is enhanced as compared to the selectivity obtained from a conventional chemotherapy.

In certain embodiments, provided herein are methods of treating cancer while reducing the adverse effects associated with administration of a chemotherapeutic agent in a patient, comprising administering to the patient in need thereof a therapeutically effective amount of a compound, wherein the compound is 4-amino-2-(2,6-dioxo-piperidine-3-yl)-isoindoline-1,3-dione, an enantiomer, a mixture of enantiomers, a pharmaceutically acceptable salt, solvate, hydrate, co-crystal, clathrate, or polymorph thereof as a single agent or as a part of a combination therapy.

As used herein, “selectivity” refers to preferential distribution of 4-amino-2-(2,6-dioxo-piperidine-3-yl)-isoindoline-1,3-dione or an enantiomer or a mixture of enantiomers thereof, or a pharmaceutically acceptable salt, solvate, hydrate, co-crystal, clathrate, or polymorph thereof in cancer cells. For example, 4-amino-2-(2,6-dioxo-piperidine-3-yl)-isoindoline-1,3-dione or an enantiomer or a mixture of enantiomers thereof, or a pharmaceutically acceptable salt, solvate, hydrate, co-crystal, clathrate, or polymorph thereof when administered to a cancer patient distributes such that the rate of distribution of the compound to cancer cells is 2- to 10-fold higher as compared to the rate of distribution of the compound to healthy cells.

As used herein, “selectively targeting” or “enhancing selectivity” refers to distribution of 4-amino-2-(2,6-dioxo-piperidine-3-yl)-isoindoline-1,3-dione or an enantiomer or a mixture of enantiomers thereof, or a pharmaceutically acceptable salt, solvate, hydrate, co-crystal, clathrate, or polymorph thereof in a cancer patient, such that the rate of distribution of the compound to cancer cells is 2- to 10-fold higher as compared to the rate of distribution of the compound to healthy cells.

In certain embodiments, the rate of distribution of pomalidomide to cancer cells is 4- to 10-fold, 2- to 8-fold, 4- to 8-fold or 2- to 6-fold higher as compared to healthy cells.

As used herein, “adverse effects” refer to the undesired side effects of chemotherapeutic agents, such as drowsiness, somnolence, dizziness, orthostatic hypotension, neutropenia, infections that result from neutropenia, increased HIV-viral load, bradycardia, Stevens-Johnson Syndrome, toxic epidermal necrolysis, and seizures (e.g., grand mal convulsions).

As used herein, unless otherwise specified, the term “treating” refers to the administration of a compound, or other additional active agent, after the onset of symptoms of the particular cancer. As used herein, unless otherwise specified, the term “preventing” refers to the administration prior to the onset of symptoms, particularly to patients at risk of cancer. The term “prevention” includes the inhibition of a symptom of the particular cancer. In certain embodiments, patients with familial history of cancer in particular are candidates for preventive regimens. As used herein and unless otherwise indicated, the term “managing” encompasses preventing the recurrence of the particular cancer in a patient who had suffered from it, lengthening the time a patient who had suffered from the cancer remains in remission, and/or reducing mortality rates of the patients.

The term “relapsed” refers to a situation where patients who have had a remission of cancer after therapy have a return of a cancerous condition. The term “refractory or resistant” refers to a circumstance where patients, even after intensive treatment, have a residual cancerous condition.

As used herein, “conventional therapy” includes, but is not limited to, surgery, immunotherapy, biological therapy, chemotherapy, radiation therapy, or other non-drug based therapy presently used to treat, prevent or manage cancer.

In certain embodiments, provided herein are pharmaceutical compositions, single unit dosage forms, and dosing regimens which comprise 4-amino-2-(2,6-dioxo-piperidine-3-yl)-isoindoline-1,3-dione, or a pharmaceutically acceptable salt, solvate (e.g., hydrate), stereoisomer, clathrate, or prodrug thereof, and a second, or additional, active agent or ingredient. Second active agents or ingredients include specific combinations, or “cocktails,” of drugs or therapy, or both.

In one embodiment, 4-amino-2-(2,6-dioxo-piperidine-3-yl)-isoindoline-1,3-dione is administered in combination with another drug (“second active agent or ingredient”) or another therapy for treating, managing, or preventing cancer. Second active agents include small molecules and large molecules (e.g., proteins and antibodies), examples of which are provided herein, as well as stem cells or cord blood. Methods, or therapies, that can be used in combination with the administration of 4-amino-2-(2,6-dioxo-piperidine-3-yl)-isoindoline-1,3-dione provided herein include, but are not limited to, surgery, chemotherapy, immunotherapy, biological therapy, radiation therapy, and other non-drug based therapies presently used to treat, prevent or manage cancer.

Provided herein are pharmaceutical compositions (e.g., single unit dosage forms) that can be used in methods disclosed herein. Particular pharmaceutical compositions comprise 4-amino-2-(2,6-dioxo-piperidine-3-yl)-isoindoline-1,3-dione, or a pharmaceutically acceptable salt, solvate (e.g., hydrate), stereoisomer, clathrate, or prodrug thereof, and a second active agent or ingredient.

### 6.1 Pomalidomide

Pomalidomide (also known as Pomalyst®), which was previously referred to as CC-4047, and has a chemical name of 4-amino-2-(2,6-dioxo-piperidine-3-yl)isoindoline-1,3-dione. Pomalidomide is a compound that inhibits, for example, LPS induced monocyte TNFα, IL-1B, IL-12, IL-6, MIP-1, MCP-1, GM-CSF, G-CSF, and COX-2 production, and may be used in treating various disorders. See, e.g., U.S. Pat. Nos. 5,635,517, 6,316,471, 6,476,052, 7,393,863, 7,629,360, and 7,863,297; and U.S. Patent Application Publication Nos. 2005/0143420, 2006/0166932, 2006/0188475, 2007/0048327, 2007/0066512, 2007/0155791, 2008/0051431, 2008/0317708, 2009/0087407, 2009/0088410, 2009/01317385, 2009/0148853, 2009/0232776, 2009/0232796, 2010/0098657, 2010/0099711, and 2011/0184025, the entireties of which are incorporated herein by reference. The compound is also known to co-stimulate the activation of T-cells. Pomalidomide has direct anti-myeloma tumoricidal activity, immunomodulatory activities and inhibits stromal cell support for multiple myeloma tumor cell growth. Specifically, pomalidomide inhibits proliferation and induces apoptosis of hematopoietic tumor cells. Id. Additionally, Pomalidomide inhibits the proliferation of lenalidomide-resistant multiple myeloma cell lines and synergizes with dexamethasone in both lenalidomide-sensitive and lenalidomide-resistant cell lines to induce tumor cell apoptosis. Pomalidomide enhances T cell- and natural killer (“NK”) cell-mediated immunity, and inhibits production of pro-inflammatory cytokines (e.g., TNF-α and IL-6) by monocytes. Pomalidomide also inhibits angiogenesis by blocking the migration and adhesion of endothelial cells. Due to its diversified pharmacological properties, Pomalidomide is useful in treating, preventing, and/or managing various diseases or disorders.

Pomalidomide and methods of synthesizing the compound are described, e.g., in U.S. Pat. Nos. 5,635,517, 6,335,349, 6,316,471, 6,476,052, 7,041,680, 7,709,502, and 7,994,327; and U.S. Patent Application Publication Nos. 2006/0178402 and 2011/0224440; the entireties of which are incorporated herein by reference.

As used herein, and unless otherwise indicated, the compound referred to herein as “Pomalidomide,” “CC-4047,” “4-amino-2-(2,6-dioxo-piperidine-3-yl)-isoindoline-1,3-dione,” or “POM” may be used herein to refer to, but not limited to, either a free base, pharmaceutically acceptable salt, solvate, hydrate, polymorph, isotopologue, deuterated derivative, co-crystal, prodrug, stereoisomer, racemate, enantiomer, and the like.

Unless otherwise specified, the terms “solid form,” “solid forms,” and related terms, when used herein to refer to Pomalidomide, refer to a physical form comprising Pomalidomide, which is not predominantly in a liquid or a gaseous state. As used herein, the terms “solid form” and “solid forms” encompass semi-solids. Solid forms may be crystalline, amorphous, partially crystalline, partially amorphous, or mixtures of forms. A “single-component” solid form comprising Pomalidomide consists essentially of Pomalidomide. A “multiple-component” solid form comprising Pomalidomide comprises a significant quantity of one or more additional species, such as ions and/or molecules, within the solid form. For example, in particular embodiments, a crystalline multiple-component solid form comprising Pomalidomide further comprises one or more species non-covalently bonded at regular positions in the crystal lattice.

Unless otherwise specified, the term “crystalline” and related terms used herein, when used to describe a substance, component, product, or form, mean that the substance, component, product, or form is substantially crystalline, for example, as determined by X-ray diffraction. (see, e.g., Remington's Pharmaceutical Sciences, 18th ed., Mack Publishing, Easton Pa., 173 (1990); The United States Pharmacopeia, 23rd ed., 1843-1844 (1995)).

Unless otherwise specified, the term “crystal form,” “crystal forms,” and related terms herein refer to crystalline modifications comprising a given substance, including single-component crystal forms and multiple-component crystal forms, and including, but not limited to, polymorphs, solvates, hydrates, co-crystals, other molecular complexes, salts, solvates of salts, hydrates of salts, co-crystals of salts, and other molecular complexes of salts, and polymorphs thereof. In some embodiments, a crystal form of a substance may be substantially free of amorphous forms and/or other crystal forms. In other embodiments, a crystal form of a substance may contain less than about 1%, 2%, 3%, 4%, 5%, 10%, 15%, 20%, 25%, 30%, 35%, 40%, 45%, or 50% of one or more amorphous form(s) and/or other crystal form(s) on a weight basis. Crystal forms of a substance may be obtained by a number of methods. Such methods include, but are not limited to, melt recrystallization, melt cooling, solvent recrystallization, recrystallization in confined spaces such as, e.g., in nanopores or capillaries, recrystallization on surfaces or templates such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding, and solvent-drop grinding.

Unless otherwise specified, the terms “polymorph,” “polymorphic form,” “polymorphs,” “polymorphic forms,” and related terms herein refer to two or more crystal forms that consist essentially of the same molecule, molecules or ions. Different polymorphs may have different physical properties, such as, for example, melting temperatures, heats of fusion, solubilities, dissolution rates, and/or vibrational spectra as a result of a different arrangement or conformation of the molecules or ions in the crystal lattice. The differences in physical properties exhibited by polymorphs may affect pharmaceutical parameters, such as storage stability, compressibility and density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically a more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing; for example, one polymorph might be more likely to form solvates or might be difficult to filter and wash free of impurities (e.g., particle shape and size distribution might be different between polymorphs). In exemplary embodiments, provided herein are solid forms of Pomalidomide, as disclosed in International Application No. PCT/US2013/026662, filed Feb. 19, 2013 and U.S. Provisional Application No. 61/805,444, filed Mar. 26, 2013, which are incorporated by reference herein in their entirety.

Unless otherwise specified, the term “cocrystal” or “co-crystal,” as used herein, refers to a crystalline material comprised of two or more non-volative compounds bond together in a crystal lattice by non-covalent interactions.

Unless otherwise specified, the term “pharmaceutical co-crystal” or “co-crystal” of an active pharmaceutical ingredient (“API”), as used herein, refers to a crystalline material comprised of an API and one or more non-volative compound(s) (referred herein as a coformer). The API and the coformer interact through non-covalent forces in a crystal lattice. In exemplary embodiments, provided herein are co-crystals of Pomalidomide, as disclosed in U.S. Provisional Application No. 61/805,444, filed Mar. 26, 2013, which is incorporated by reference herein in its entirety. In one embodiment, provided herein are solid forms (e.g., co-crystals) of Pomalidomide.

Unless otherwise specified, the term “amorphous,” “amorphous form,” and related terms used herein mean that the substance, component, or product referred to is not substantially crystalline as determined by X-ray diffraction. In certain embodiments, an amorphous form of a substance may be substantially free of crystal forms. In other embodiments, an amorphous form of a substance may contain less than about 1%, 2%, 3%, 4%, 5%, 10%, 15%, 20%, 25%, 30%, 35%, 40%, 45% or 50% of one or more crystal forms on a weight basis. In other embodiments, an amorphous form of a substance may comprise additional components or ingredients (for example, an additive, a polymer, or an excipient that may serve to further stabilize the amorphous form). In some embodiments, amorphous form may be a solid solution. Amorphous forms of a substance can be obtained by a number of methods. Such methods include, but are not limited to, heating, melt cooling, rapid melt cooling, solvent evaporation, rapid solvent evaporation, desolvation, sublimation, grinding, ball-milling, cryo-grinding, spray drying, and freeze drying.

The compounds provide herein may also contain an unnatural proportion of an atomic isotope at one or more of the atoms that constitute such a compound. For example, the compound may be radiolabeled with radioactive isotopes, such as for example tritium (³H), iodine-125 (¹²⁵I) sulfur-35 (³⁵S), or carbon-14 (¹⁴C). Radiolabeled compounds are useful as therapeutic agents, e.g., cancer therapeutic agents, research reagents, e.g., binding assay reagents, and diagnostic agents, e.g., in vivo imaging agents. All isotopic variations of the compounds provided herein, whether radioactive or not, are intended to be encompassed herein. In certain embodiments, a compound provided herein contains unnatural proportion(s) of one or more isotopes, including, but not limited to, hydrogen (¹H), deuterium (²H), tritium (³H), carbon-11 (¹¹C), carbon-12 (¹²C), carbon-13 (13C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸P), fluorine-17 (¹⁷F), fluorine-18 (F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 ¹²³I) iodine-125I) iodine-127 (¹²⁷I), iodine-129 (¹²⁹I), and iodine-131 (¹³¹I). In certain embodiments, a compound provided herein contains unnatural proportion(s) of one or more isotopes in a stable form, that is, non-radioactive, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, a compound provided herein contains unnatural proportion(s) of one or more isotopes in an unstable form, that is, radioactive, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I) iodine-125 (¹²⁵I), iodine-129 (¹²⁹I), and iodine-131 (¹³¹I). In certain embodiments, in a compound as provided herein, any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, a compound provided herein contains unnatural proportions of deuterium (“D”). In exemplary embodiments, provided herein are isotopologues of Pomalidomide, as disclosed in U.S. Provisional Application No. 61/500,053, filed Jun. 22, 2011, which is incorporated by reference herein in its entirety. In one embodiment, provided herein are solid forms (e.g., crystal forms, amorphous forms, or mixtures thereof) of isotopologues of Pomalidomide provided herein.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of 4-amino-2-(2,6-dioxo-piperidine-3-yl)-isoindoline-1,3-dione that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of 4-amino-2-(2,6-dioxo-piperidine-3-yl)-isoindoline-1,3-dione that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

4-amino-2-(2,6-dioxo-piperidine-3-yl)-isoindoline-1,3-dione contains a chiral center, and thus can exist as a racemic mixture of R and S enantiomers. Provided herein is the use of stereomerically pure forms of this compound, as well as the use of mixtures of those forms. For example, mixtures comprising equal or unequal amounts of the enantiomers may be used in methods and compositions. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, or greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. As used herein and unless otherwise indicated, the term “stereomerically enriched” means a composition that comprises greater than about 60% by weight of one stereoisomer of a compound, or greater than about 70% by weight, or greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center. In other words, encompassed is the use of the R or S enantiomer of 4-amino-2-(2,6-dioxo-piperidine-3-yl)-isoindoline-1,3-dione in the methods.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

Citation of any references in this Section is not to be construed as an admission that such references are prior art to the present application.

### 6.2 Methods

Methods provided herein encompass those for treating, preventing or managing various types cancers. The cancers include but not limited to bladder cancer, breast cancer, cervical cancer, colon cancer (including colorectal cancer), esophageal cancer, head and neck cancer, liver cancer, lung cancer (both small cell and non-small cell), melanoma, ovarian cancer, pancreatic cancer, prostate cancer, renal cancer, sarcoma (including osteosarcoma), skin cancer (including squamous cell carcinoma), stomach cancer, testicular cancer, thyroid cancer, and uterine cancer. In a specific embodiment, the cancer is metastatic.

In one embodiment, the cancer is refractory, relapsed, or is resistant to chemotherapy other than 4-amino-2-(2,6-dioxo-piperidine-3-yl)-isoindoline-1,3-dione. In one embodiment, the cancer is resistant to chemotherapy with lenalidomide.

In certain embodiments, provided herein are methods of selectively targeting cancer cells, while leaving healthy cells intact, in a patient comprising administering to the patient in need thereof a therapeutically effective amount of a compound, wherein the compound is 4-amino-2-(2,6-dioxo-piperidine-3-yl)-isoindoline-1,3-dione, an enantiomer, a mixture of enantiomers, a pharmaceutically acceptable salt, solvate, hydrate, co-crystal, clathrate, or polymorph thereof as a single agent or as a part of a combination therapy.

In certain embodiments, provided herein are methods of enhancing selectivity against cancer cells as compared to healthy cells in a patient comprising administering to the patient in need thereof a therapeutically effective amount of a compound, wherein the compound is 4-amino-2-(2,6-dioxo-piperidine-3-yl)-isoindoline-1,3-dione, an enantiomer, a mixture of enantiomers, a pharmaceutically acceptable salt, solvate, hydrate, co-crystal, clathrate, or polymorph thereof as a single agent or as a part of a combination therapy, wherein selectivity is enhanced as compared to the selectivity obtained from a conventional chemotherapy.

In one embodiment, in the methods provided herein the rate of distribution of pomalidomide to cancer cells is 2- to 10-fold higher as compared to the rate of distribution of pomalidomide to healthy cells. In another embodiment, the rate of distribution of pomalidomide to cancer cells is 4- to 10-fold higher as compared to healthy cells. In another embodiment, the rate of distribution of pomalidomide to cancer cells is 2- to 8-fold higher as compared to healthy cells. In another embodiment, the rate of distribution of pomalidomide to cancer cells is 4- to 8-fold higher as compared to healthy cells. In another embodiment, the rate of distribution of pomalidomide to cancer cells is 2- to 6-fold higher as compared to healthy cells. In another embodiment, the rate of distribution of pomalidomide to cancer cells is 2, 3, 4, 5, 6, 7, 8, or 10-fold higher as compared to healthy cells.

In certain embodiments, provided herein are methods of treating cancer while reducing the adverse effects associated with administration of a chemotherapeutic agent in a patient, comprising administering to the patient in need thereof a therapeutically effective amount of a compound, wherein the compound is 4-amino-2-(2,6-dioxo-piperidine-3-yl)-isoindoline-1,3-dione, an enantiomer, a mixture of enantiomers, a pharmaceutically acceptable salt, solvate, hydrate, co-crystal, clathrate, or polymorph thereof as a single agent or as a part of a combination therapy. The chemotherapeutic agents are described elsewhere herein.

In certain embodiments, the adverse effects are drowsiness, somnolence, dizziness, orthostatic hypotension, neutropenia, infections that result from neutropenia, increased HIV-viral load, bradycardia, Stevens-Johnson Syndrome, toxic epidermal necrolysis, and seizures (e.g., grand mal convulsions). In one embodiment, the adverse effect is neutropenia.

Without being limited by theory, it is believed that the cancer cell targeting property of 4-amino-2-(2,6-dioxo-piperidine-3-yl)-isoindoline-1,3-dione provides augmented efficacy and minimize adverse side effects.

In the methods of treating cancer provided herein, 4-amino-2-(2,6-dioxo-piperidine-3-yl)-isoindoline-1,3-dione exhibits unique pharmacokinetic properties in cancer patients as compared to healthy subjects. In one embodiment, apparent peripheral volume of distribution (V3/F) of 4-amino-2-(2,6-dioxo-piperidine-3-yl)-isoindoline-1,3-dione in cancer patients is about 2 to 10-fold higher than that in healthy subjects. In one embodiment, apparent peripheral volume of distribution (V3/F) of 4-amino-2-(2,6-dioxo-piperidine-3-yl)-isoindoline-1,3-dione in cancer patients is about 2 to 8-fold higher than that in healthy subjects. In one embodiment, apparent peripheral volume of distribution (V3/F) of 4-amino-2-(2,6-dioxo-piperidine-3-yl)-isoindoline-1,3-dione in cancer patients is about 2, 4, 6, 8 or 10-fold higher than that in healthy subjects.

In one embodiment, apparent intercompartmental clearance (Q/F) of 4-amino-2-(2,6-dioxo-piperidine-3-yl)-isoindoline-1,3-dione in cancer patients is about 1 to 6-fold higher than that in healthy subjects. In one embodiment, apparent intercompartmental clearance (Q/F) of 4-amino-2-(2,6-dioxo-piperidine-3-yl)-isoindoline-1,3-dione in cancer patients is about 2 to 4-fold higher than that in healthy subjects. In one embodiment, apparent intercompartmental clearance (Q/F) of 4-amino-2-(2,6-dioxo-piperidine-3-yl)-isoindoline-1,3-dione in cancer patients is about 2, 3 or 4-fold higher than that in healthy subjects.

Without being limited by theory, it is believed that once 4-amino-2-(2,6-dioxo-piperidine-3-yl)-isoindoline-1,3-dione is in blood circulation, it naturally accumulates in cancer cells but not in healthy cells.

Provided herein are methods of treating patients who have been previously treated for cancer, but are non-responsive to standard therapies, as well as those who have not previously been treated. Also, provided herein are methods of treating patients regardless of patient's age, although some cancers are more common in certain age groups. Further, herein provided are methods of treating patients who have undergone surgery in an attempt to treat the cancer at issue, as well as those who have not. Because patients with cancer have heterogenous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

In one embodiment, 4-amino-2-(2,6-dioxo-piperidine-3-yl)-isoindoline-1,3-dione is administered orally and in single or divided daily doses in an amount from about 0.10 to about 150 mg/day. In another embodiment, 4-amino-2-(2,6-dioxo-piperidine-3-yl)-isoindoline-1,3-dione is administered in an amount from about 0.10 to 100 mg per day, from about 0.5 to about 50 mg per day, or from about 1 to about 10 mg per day. Specific doses per day include 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In one embodiment, the recommended starting dose of 4-amino-2-(2,6-dioxo-piperidine-3-yl)-isoindoline-1,3-dione is 0.5 mg per day. The dose can be escalated every week to 1, 1.5, 2, 2.5, 3, 3.5, 4, 4.5, 5 and 10 mg per day. The patients who are dosed initially at 4 mg and who experience thrombocytopenia or neutropenia that develops within or after the first four weeks of starting 4-amino-2-(2,6-dioxo-piperidine-3-yl)-isoindoline-1,3-dione therapy may have their dosage adjusted according to a platelet count or absolute neutrophil count (“ANC”).

### 6.2.1 Combination Therapy with a Second Active Agent

In one embodiment, the methods provided herein comprise administering 4-amino-2-(2,6-dioxo-piperidine-3-yl)-isoindoline-1,3-dione, or a pharmaceutically acceptable salt, solvate (e.g., hydrate), stereoisomer, clathrate, or prodrug thereof, in combination with one or more second active agents, and/or in combination with radiation therapy, blood transfusions, or surgery.

It is believed that certain combinations work synergistically in the treatment of particular types of cancer. 4-amino-2-(2,6-dioxo-piperidine-3-yl)-isoindoline-1,3-dione can also work to alleviate adverse effects associated with certain second active agents, and some second active agents can be used to alleviate adverse effects associated with 4-amino-2-(2,6-dioxo-piperidine-3-yl)-isoindoline-1,3-dione.

Examples of 4-amino-2-(2,6-dioxo-piperidine-3-yl)-isoindoline-1,3-dione are disclosed herein (see, e.g., section 5.1). Second active agents or chemotherapeutic agent can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Typical large molecule active agents are biological molecules, such as naturally occurring or artificially made proteins. Proteins that are particularly useful include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

Particular proteins that can be used in the methods and include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; all of which are incorporated herein by reference.

Provided herein are the use of native, naturally occurring, and recombinant proteins. Provided herein are mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with compounds provided herein include monoclonal and polyclonal antibodies. Examples of antibodies include, but are not limited to, trastuzumab (Herceptin®), rituximab (Rituxan®), bevacizumab (Avastin™), pertuzumab (Omnitarg™), tositumomab (Bexxar®), edrecolomab (Panorex®), and G250. Compounds provided herein can also be combined with, or used in combination with, anti-TNF-αantibodies.

Large molecule active agents may be administered in the form of anti-cancer vaccines. For example, vaccines that secrete, or cause the secretion of, cytokines such as IL-2, G-CSF, and GM-CSF can be used in the methods, pharmaceutical compositions, and kits. See, e.g., Emens, L. A., et al., Curr. Opinion Mol. Ther. 3(1):77-84 (2001).

In one embodiment provided herein, the large molecule active agent reduces, eliminates, or prevents an adverse effect associated with the administration of 4-amino-2-(2,6-dioxo-piperidine-3-yl)-isoindoline-1,3-dione. Depending on the disease or disorder begin treated, adverse effects can include, but are not limited to, drowsiness and somnolence, dizziness and orthostatic hypotension, neutropenia, infections that result from neutropenia, increased HIV-viral load, bradycardia, Stevens-Johnson Syndrome and toxic epidermal necrolysis, and seizures (e.g., grand mal convulsions).

Second active agents that are small molecules can also be used to alleviate adverse effects associated with the administration of 4-amino-2-(2,6-dioxo-piperidine-3-yl)-isoindoline-1,3-dione. However, like some large molecules, many are believed to be capable of providing a synergistic effect when administered with (e.g., before, after or simultaneously) 4-amino-2-(2,6-dioxo-piperidine-3-yl)-isoindoline-1,3-dione. Examples of small molecule second active agents include, but are not limited to, anti-cancer agents, antibiotics, immunosuppressive agents, and steroids.

Examples of anti-cancer agents include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; bortezomib (Velcade®); brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®); imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim;Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents include, but are not limited to, rituximab, bortezomib, oblimersen)(Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

Administration of 4-amino-2-(2,6-dioxo-piperidine-3-yl)-isoindoline-1,3-dione and the second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. In one embodiment, 4-amino-2-(2,6-dioxo-piperidine-3-yl)-isoindoline-1,3-dione is administered orally. Routes of administration for the second active agents or ingredients provided herein are known to those of ordinary skill in the art. See, e.g., Physicians' Desk Reference, (2006).

In one embodiment, the second active agent is administered intravenously or subcutaneously and once or twice daily in an amount of from about 1 to about 1,000 mg, from about 5 to about 500 mg, from about 10 to about 375 mg, or from about 50 to about 200 mg. The specific amount of the second active agent will depend on the specific agent used, the type of disease being treated or managed, the severity and stage of disease, and the amount(s) of 4-amino-2-(2,6-dioxo-piperidine-3-yl)-isoindoline-1,3-dione and any optional additional active agents concurrently administered to the patient. In a particular embodiment, the second active agent is rituximab, bortezomib, oblimersen (Genasense®), GM-CSF, G-CSF, EPO, taxotere, irinotecan, dacarbazine, transretinoic acid, topotecan, pentoxifylline, ciprofloxacin, dexamethasone, vincristine, doxorubicin, COX-2 inhibitor, IL2, IL8, IL18, IFN, Ara-C, vinorelbine, or a combination thereof.

In another embodiment, provided herein are methods for treating, preventing and/or managing cancer, which comprises administering 4-amino-2-(2,6-dioxo-piperidine-3-yl)-isoindoline-1,3-dione provided herein, or a pharmaceutically acceptable salt, solvate (e.g., hydrate), stereoisomer, clathrate, or prodrug thereof, in conjunction with (e.g., before, during, or after) conventional therapy including, but not limited to, surgery, immunotherapy, biological therapy, chemotherapy of other anti cancer agents, radiation therapy, or other non-drug based therapy presently used to treat, prevent or manage cancer. The combined use of the 4-amino-2-(2,6-dioxo-piperidine-3-yl)-isoindoline-1,3-dione provided herein and conventional therapy may provide a unique treatment regimen that is unexpectedly effective in certain patients. Without being limited by theory, it is believed that 4-amino-2-(2,6-dioxo-piperidine-3-yl)-isoindoline-1,3-dione may provide additive or synergistic effects when given concurrently with conventional therapy.

In one embodiment, provided herein are methods of reducing, treating and/or preventing adverse or undesired effects associated with conventional therapy including, but not limited to, surgery, chemotherapy, radiation therapy, hormonal therapy, biological therapy and immunotherapy. 4-amino-2-(2,6-dioxo-piperidine-3-yl)-isoindoline-1,3-dione and other active ingredients can be administered to a patient prior to, during, or after the occurrence of the adverse effect associated with conventional therapy.

In one embodiment, 4-amino-2-(2,6-dioxo-piperidine-3-yl)-isoindoline-1,3-dione can be administered in an amount of from about 0.10 to about 150 mg, or from about 0.5 to about 50 mg, or from about 1 to about 25 mg orally and daily alone, or in combination with a second active agent disclosed herein (see, e.g., section 5.2), prior to, during, or after the use of conventional therapy.

### 6.2.2 Use with Transplantation Therapy

Compounds provided herein can be used to reduce the risk of Graft Versus Host Disease (“GVHD”). Therefore, provided herein are methods of treating, preventing and/or managing cancer, which comprises administering 4-amino-2-(2,6-dioxo-piperidine-3-yl)-isoindoline-1,3-dione, or a pharmaceutically acceptable salt, solvate (e.g., hydrate), stereoisomer, clathrate, or prodrug thereof, in conjunction with transplantation therapy.

As those of ordinary skill in the art are aware, the treatment of cancer is often based on the stages and mechanism of the disease. For example, as inevitable leukemic transformation develops in certain stages of cancer, transplantation of peripheral blood stem cells, hematopoietic stem cell preparation or bone marrow may be necessary. The combined use of 4-amino-2-(2,6-dioxo-piperidine-3-yl)-isoindoline-1,3-dione and transplantation therapy provides a unique and unexpected synergism. In particular, 4-amino-2-(2,6-dioxo-piperidine-3-yl)-isoindoline-1,3-dione exhibits immunomodulatory activity that may provide additive or synergistic effects when given concurrently with transplantation therapy in patients with cancer.

4-amino-2-(2,6-dioxo-piperidine-3-yl)-isoindoline-1,3-dione can work in combination with transplantation therapy reducing complications associated with the invasive procedure of transplantation and risk of GVHD. Provided herein are methods for treating, preventing and/or managing cancer which comprises administering to a patient (e.g., a human) 4-amino-2-(2,6-dioxo-piperidine-3-yl)-isoindoline-1,3-dione, or a pharmaceutically acceptable salt, solvate (e.g., hydrate), stereoisomer, clathrate, or prodrug thereof, before, during, or after the transplantation of umbilical cord blood, placental blood, peripheral blood stem cell, hematopoietic stem cell preparation or bone marrow. Examples of stem cells suitable for use in the methods provided herein are disclosed in U.S. patent publication nos. 2002/0123141, 2003/0235909 and 2003/0032179, by R. Hariri et al., the entireties of which are incorporated herein by reference.

### 6.2.3 Cycling Therapy

In certain embodiments, the prophylactic or therapeutic agents provided herein are cyclically administered to a patient. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

Consequently, in one specific embodiment, 4-amino-2-(2,6-dioxo-piperidine-3-yl)-isoindoline-1,3-dione is administered daily in a single or divided doses in a four to six week cycle with a rest period of about a week or two weeks. The embodiment further allows the frequency, number, and length of dosing cycles to be increased. Thus, another specific embodiment provided herein encompasses the administration of 4-amino-2-(2,6-dioxo-piperidine-3-yl)-isoindoline-1,3-dione for more cycles than are typical when it is administered alone. In yet another specific embodiment, 4-amino-2-(2,6-dioxo-piperidine-3-yl)-isoindoline-1,3-dione is administered for a greater number of cycles that would typically cause dose-limiting toxicity in a patient to whom a second active ingredient is not also being administered.

In one embodiment, 4-amino-2-(2,6-dioxo-piperidine-3-yl)-isoindoline-1,3-dione is administered daily and continuously for three or four weeks at a dose of from about 0.10 to about 150 mg/d followed by a break of one or two weeks. In a particular embodiment, 4-amino-2-(2,6-dioxo-piperidine-3-yl)-isoindoline-1,3-dione is administered in an amount of from about 1 to about 50 mg/day, or in an amount of about 4 mg/day for three to four weeks, followed by one week or two weeks of rest in a four or six week cycle.

In one embodiment, 4-amino-2-(2,6-dioxo-piperidine-3-yl)-isoindoline-1,3-dione is administered to patients with various types of cancer, in an amount of about 0.5 mg, 1 mg, 2 mg, 3 mg or 4 mg per day for 21 days followed by seven days rest in a 28 day cycle. In another embodiment, 4-amino-2-(2,6-dioxo-piperidine-3-yl)-isoindoline-1,3-dione is administered to patients with refractory or relapsed cancers in an amount of about 4 mg per day for 21 days followed by seven days rest in a 28 day cycle.

In one embodiment, 4-amino-2-(2,6-dioxo-piperidine-3-yl)-isoindoline-1,3-dione and a second active agent or ingredient are administered orally, with administration of 4-amino-2-(2,6-dioxo-piperidine-3-yl)-isoindoline-1,3-dione occurring 30 to 60 minutes prior to a second active ingredient, during a cycle of four to six weeks. In another embodiment, 4-amino-2-(2,6-dioxo-piperidine-3-yl)-isoindoline-1,3-dione is administered orally and a second active ingredient is administered by intravenous infusion.

In a specific embodiment, one cycle comprises the administration of from about 0.1 to about 25 mg/day of 4-amino-2-(2,6-dioxo-piperidine-3-yl)-isoindoline-1,3-dione and from about 50 to about 750 mg/m²/day of a second active ingredient daily for three to four weeks and then one or two weeks of rest.

In one embodiment, rituximab can be administered in an amount of 375 mg/m² as an additional active agent to patients with various types of cancer. In one embodiment, rituximab can be administered in an amount of 375 mg/m² as an additional active agent to patients with refractory or relapsed cancer. In one embodiment, one cycle comprises the administration of 4-amino-2-(2,6-dioxo-piperidine-3-yl)-isoindoline-1,3-dione given orally daily for 21 days followed by 7 days of rest and 375 mg/m² of rituximab by intravenous infusion weekly for four weeks.

Typically, the number of cycles during which the combinatorial treatment is administered to a patient will be from about one to about 24 cycles, more typically from about two to about 16 cycles, and even more typically from about four to about three cycles.

### 6.3 Pharmaceutical Compositions and Dosage Forms

Pharmaceutical compositions can be used in the preparation of individual, single unit dosage forms. Pharmaceutical compositions and dosage forms provided herein comprise 4-amino-2-(2,6-dioxo-piperidine-3-yl)-isoindoline-1,3-dione, or a pharmaceutically acceptable salt, solvate (e.g., hydrate), stereoisomer, clathrate, or prodrug thereof. Pharmaceutical compositions and dosage forms provided herein can further comprise one or more recipients.

Pharmaceutical compositions and dosage forms provided herein can also comprise one or more additional active ingredients. Consequently, pharmaceutical compositions and dosage forms provided herein comprise the active ingredients disclosed herein (e.g., 4-amino-2-(2,6-dioxo-piperidine-3-yl)-isoindoline-1,3-dione and a second active agent). Examples of optional second, or additional, active ingredients are disclosed herein (see, e.g., section 5.2).

Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms provided herein will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed herein will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more recipients. Suitable recipients are well known to those skilled in the art of pharmacy, and non-limiting examples of suitable recipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain recipients not suited for use in parenteral dosage forms. The suitability of a particular excipient may also depend on the specific active ingredients in the dosage form. For example, the decomposition of some active ingredients may be accelerated by some recipients such as lactose, or when exposed to water. Active ingredients that comprise primary or secondary amines are particularly susceptible to such accelerated decomposition. Consequently, provided herein are pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions provided herein can comprise recipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. In one embodiment, lactose-free dosage forms comprise active ingredients, microcrystalline cellulose, pre-gelatinized starch, and magnesium stearate.

Further provided herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles& Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are, in certain embodiments, anhydrous if substantial contact with moisture and/or humidity during manufacturing, packaging, and/or storage is expected.

An anhydrous pharmaceutical composition should be prepared and stored such that its anhydrous nature is maintained. Accordingly, in one embodiment, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Provided herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of recipients, the amounts and specific types of active ingredients in a dosage form may differ depending on factors such as, but not limited to, the route by which it is to be administered to patients. However, typical dosage forms provided herein comprise 4-amino-2-(2,6-dioxo-piperidine-3-yl)-isoindoline-1,3-dione or a pharmaceutically acceptable salt, solvate (e.g., hydrate), stereoisomer, clathrate, or prodrug thereof in an amount of from about 0.10 to about 150 mg. Typical dosage forms comprise 4-amino-2-(2,6-dioxo-piperidine-3-yl)-isoindoline-1,3-dione or a pharmaceutically acceptable salt, solvate (e.g., hydrate), stereoisomer, clathrate, or prodrug thereof in an amount of about 0.1, 1, 1.5, 2, 2.5, 3, 4, 5, 7.5, 10, 12.5, 15, 17.5, 20, 25, 50, 100, 150 or 200 mg. In a specific embodiment, dosage form comprises 4-amino-2-(2,6-dioxo-piperidine-3-yl)-isoindoline-1,3-dione in an amount of about 0.1, 0.5, 1, 2.5, 3, 4, 5, 7.5, 10, 15, 20, 25 or 50 mg. Typical dosage forms comprise the second active ingredient in an amount of 1 to about 1000 mg, from about 5 to about 500 mg, from about 10 to about 350 mg, or from about 50 to about 200 mg. Of course, the specific amount of the anti-cancer drug will depend on the specific agent used, the type of cancer being treated or managed, and the amount(s) of 4-amino-2-(2,6-dioxo-piperidine-3-yl)-isoindoline-1,3-dione and any optional additional active agents concurrently administered to the patient.

### 6.3.1 Oral Dosage Forms

Pharmaceutical compositions provided herein that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

In one embodiment, a dosage form is a capsule or tablet comprising 4-amino-2-(2,6-dioxo-piperidine-3-yl)-isoindoline-1,3-dione in an amount of about 0.5, 1, 2, 3, 4, 5, 10, 15, 20, 25 or 50 mg. In a specific embodiment, a capsule or tablet dosage form comprises 4-amino-2-(2,6-dioxo-piperidine-3-yl)-isoindoline-1,3-dione in an amount of about 1, 2, 3 or 4 mg.

Typical oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, recipients suitable for use in oral liquid or aerosol dosage forms include, but are not limited to, water, glycols, oils, alcohols, flavoring agents, preservatives, and coloring agents. Examples of recipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid recipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of recipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture recipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions provided herein is typically present in from about 50 to about 99 weight percent of the pharmaceutical composition or dosage form.

Disintegrants are used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions comprise from about 0.5 to about 15 weight percent of disintegrant, or from about 1 to about 5 weight percent of disintegrant.

Disintegrants that can be used in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof

Lubricants that can be used in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A solid oral dosage form provided herein comprises 4-amino-2-(2,6-dioxo-piperidine-3-yl)-isoindoline-1,3-dione, anhydrous lactose, microcrystalline cellulose, polyvinylpyrrolidone, stearic acid, colloidal anhydrous silica, and gelatin.

### 6.3.2 Delayed Release Dosage Forms

Active ingredients provided herein can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein. Thus provided herein are single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 6.3.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, in certain embodiments, parenteral dosage forms are sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms provided herein are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms provided herein. For example, cyclodextrin and its derivatives can be used to increase the solubility of 4-amino-2-(2,6-dioxo-piperidine-3-yl)-isoindoline-1,3-dione and its derivatives. See, e.g., U.S. Pat. No. 5,134,127, which is incorporated herein by reference.

### 7. EXAMPLES

Certain embodiments provided herein are illustrated by the following non-limiting example.

### 7.1 Population Pharmacokinetic Analysis Of Pomalidomide in Multiple Myeloma Patients

### 7.1.1 Introduction

Pomalidomide has been approved in the US (4 mg once daily on Days 1 to 21 of each 28-day cycle), and is indicated for patients with multiple myeloma (MM) who have received at least two prior therapies including lenalidomide and bortezomib and have demonstrated disease progression on or within 60 days of completion of the last therapy. Pomalidomide in combination with dexamethasone has been approved in the European Union (EU), and is indicated for patients with relapsed and refractory MM who have received at least two prior treatment regimens, including both lenalidomide and bortezomib, and have demonstrated disease progression on the last therapy.

Pomalidomide is absorbed with a maximum plasma concentration (Cmax) at a median time (tmax) between 2 hours and 3 hours after clinically relevant doses. Pomalidomide is >73% absorbed following administration of a single oral dose. The systemic exposure to a single dose pomalidomide as determined from the AUC increases in an approximately dose-proportional manner, whereas Cmax generally increased in a less than dose proportional manner. Based on non-compartmental analysis, mean (percent coefficient of variation [%CV]) apparent volume of distribution (Vz/F) of pomalidomide after a single dose ranges from 74 L (20%) to 138 L (30%) across a dose range of 1 mg to 10 mg. Pomalidomide is extensively metabolized via multiple metabolic pathways and is eliminated in human via multiple pathways. The geometric mean terminal half-life (t1/2) of pomalidomide was approximately 7.5 hours, and apparent clearance (CL/F) generally ranged from 6.5 to 10.8 L/hr.

Pomalidomide plus low-dose dexamethasone significantly increased progression free survival (PFS) and overall survival (OS) compared with high-dose dexamethasone when administered to refractory or relapsed and refractory MM patients who failed lenalidomide and bortezomib and have demonstrated disease progression on the last therapy. In this indication, hematologic adverse events (AE) such as neutropenia, anemia, thrombocytopenia, and non-hematologic AEs such as fatigue, constipation, diarrhea, and pneumonia were frequently reported.

A population pharmacokinetic analysis was performed on the data from six clinical studies to characterize the pomalidomide PK and associated variability.

The analyses were conducted in accordance with the principles outlined in the US Food and Drug Administration (FDA) guidance on population PK analyses (US FDA, 1999) and the European Medicines Agency (EMA) guideline on population PK reports (EMA, 2007).

### 7.1.2 Materials And Methods

### 7.1.2.1 Studies Included

Healthy normal subjects and subjects with MM treated with pomalidomide from the following six clinical studies were included in the PPK analysis:

Study 1: A phase 1, randomized, double-blind, placebo-controlled study to evaluate the safety, tolerability, and pharmacokinetics of pomalidomide (CC-4047) following multiple daily doses in healthy male subjects.

Study 2: A phase 1, open-label, randomized, 2-period, 2-way crossover study to evaluate the bioequivalence of pomalidomide (CC-4047) capsules in healthy male subjects.

Study 3: A phase lb, open-label study of the safety and efficacy of CC-4047 treatment for patients with relapsed multiple myeloma

Study 4: A phase 1/2 multi-center, randomized, open-label, dose escalation study to determine the maximum tolerated dose, safety, and efficacy of CC-4047 alone or in combination with low-dose dexamethasone in patients with relapsed and refractory multiple myeloma who have received prior treatment that includes lenalidomide and bortezomib

Study 5: A phase 3, multicenter, randomized, open-label study to compare the efficacy and safety of pomalidomide in combination with low-dose dexamethasone versus high-dose dexamethasone in subjects with refractory or relapsed and refractory multiple myeloma

Study 6: A phase 1, multicenter, open-label, dose-escalation study to determine the maximum tolerated dose (MTD) for the combination of pomalidomide, bortezomib and low-dose dexamethasone in subjects with relapsed or refractory multiple myeloma

Pomalidomide was administered orally as a solid dosage form once daily (QD) or once every other day in all studies.

All subjects who had evaluable pomalidomide concentration data and sufficient covariate data were included in the PPK analyses. Data from both intensive (Studies 1-4 and 6) and sparse PK samples (Study 5) have been included for PPK analysis dataset.

Table 1 below summarizes the number of subjects who provided PK samples, the studied dose regimens, nominal PK sampling times, and bioanalytical method for each study:

TABLE 1
Summary of Clinical Studies Included in Population PK analysis

| | | Number | | | |
|---|---|---|---|---|---|
| Study | | of PK | Pomalidomide | Assay | |
| Number | Disease | Subjects | Treatment | Performance | PK sample times |
| 1 | Healthy | 24 | 0.5, 1, 2 mg | LC-MS/MS | 0, 0.5, 1, 1.5, 2, 2.5, 3, 4, |
| | subjects | | QD for 5 days | LLOQ = 0.250 | 6, 8, 12, 24 hour on Day 1 |
| | | | | ng/mL | 0, 0.5, 1, 1.5, 2, 2.5, 3, 4, |
| | | | | | 6, 8, 12, 24, 36, 48, 72 |
| | | | | | hour on Day 5 |
| | | | | | Predose on Day 3 and 4 |
| 2 | Healthy | 72 | 3, 4 mg | LC-MS/MS | 0, 0.5, 1, 1.5, 2, 2.5, 3, |
| | subjects | | single dose | LLOQ = 0.25 | 3.5, 4, 6, 8, 12, 16, 24, 36, |
| | | | | ng/mL | 48 hour |
| 3 | Multiple | 28 | 1, 2, 5, 10 mg | LC-MS/MS | 0, 0.25, 0.5, 0.75, 1, 1.5, |
| | myeloma | | QD up to 4 | LLOQ = 0.2 | 2, 2.5, 3, 4, 6, 8, 10, 12, |
| | | | weeks | ng/mL | 18, 24, 48 hour on Day 1 |
| | | | | | 0, 0.25, 0.5, 0.75, 1, 1.5, |
| | | | | | 2, 2.5, 3, 4, 6, 8, 10, 12, |
| | | | | | 18, 24 hour on Day 28 |
| 4 | Multiple | 14 | 4 mg | LC-MS/MS | 0, 0.5, 1, 2, 3, 4, 6, 8 hour |
| | myeloma | | QD on Days | LLOQ = 0.50 | on Day 1 |
| | | | 1-21 per cycle | ng/mL | 0, 0.5, 1, 2, 3, 4, 6, 8 hour |
| | | | (28 days) | | on Day 8 |
| 5 | Multiple | 80 | 4 mg | LC-MS/MS | Predose and 2 hour post |
| | myeloma | | QD on Days | LLOQ = 0.25 | dose on C1D1, C1D8, |
| | | | 1-21 per cycle | ng/mL | C1D15 and C1D22 |
| | | | (28 days) | | |
| 6 | Multiple | 22 | 1, 2, 3, 4 mg | LC-MS/MS | 0, 1, 2, 4, 6 hour on C1D8 |
| | myeloma | | QD on Days | LLOQ = 0.25 | in dose determination |
| | | | 1-14 per cycle | ng/mL | phase, and 0, 0.5, 1, 2, 3, |
| | | | (21 days) | | 4, 6, 8, 24 hour on C1D8 |
| | | | | | in dose confirmation |
| | | | | | phase |

C = cycle; D = day; LC-MS/MS = liquid chromatography with tandem mass spectrometry; LLOQ = lower limit of quantitation; PK = pharmacokinetic; and QD = once daily.

### 7.1.2.2 Software for Data Analyses

The software applications used in the data analyses and data presentation include:
NONMEM (Version 7.2, ICON Development Solution, Md., US).
SAS (Version 9.2, SAS Institute, Inc., Cary, N.C., US)
S-PLUS (Version 8.2, TIBCO Software Inc, Somerville, Mass., US) vPerl-Speaks-NONMEM (PsN, Version 3.5.3, by Kajsa Harling and Andrew Hooker)
WinNonlin (Version 6.1, Pharsight Corporation, Mountain View, Calif., US)

The nonlinear mixed effects modeling program NONMEM was installed with Intel Visual Fortran compiler (Version 11.1, Intel Corporation, Calif., US) on Windows 7 Professional running a Quadra-core Intel Core i7 CPU server with 16 GB of memory. Population PK analyses were performed using NONMEM with the first-order conditional estimation (FOCE) using natural logarithm (Ln)-transformed concentration data. All models were compiled with Intel Visual Fortran and were run via PsN. Exploratory exposure-response analysis for efficacy and safety were performed using S-PLUS. Analysis dataset preparation, data processing, and diagnostic plots were completed using S-PLUS. Individual plasma concentration profiles were simulated through NONMEM, and various exposure metrics, such as area under concentration-time curve (AUC) and Cmax were calculated using S-PLUS:

### 7.1.2.3 Dataset Construction

### 7.1.2.3.1 Source Datasets

Pomalidomide concentrations, safety laboratory results, efficacy data, dosing records, PK sampling times, and demographic data were pooled from the respective clinical studies to form the source datasets for analysis. All source datasets were formatted in SAS transport files.

### 7.1.2.3.2 Analysis Datasets

NONMEM formatted datasets were created from the pooled datasets for PPK. Typical variables in NONMEM formatted datasets include:
PK variables: actual sampling time relative to first and previous dose, actual dose time relative to first dose, pomalidomide concentration values, natural logarithm (Ln)-transformed pomalidomide concentration values
Demographic factors: age, body weight, body surface area, sex, and race
Hepatic function markers: total bilirubin, albumin, AST, or other markers as appropriate
Renal function marker: creatinine clearance estimated by Cockcroft-Gault formula
Status of health (healthy subjects versus MM subjects)

The units of continuous variables were consistent across the studies.

The Cockcroft-Gault equation below was used to calculate creatinine clearance (CrCL):

CrCL=[(140−Age)×Body Weight]/(72×Serum Creatinine Level)×(0.85 for Females)

CrCL were capped to a physiological level of 150 mL/min

All analysis datasets were constructed using S-PLUS scripts in order to ensure a full traceability of the processes of analysis dataset construction.

### 7.1.2.3.3 Data Handling

Major steps of data compilation and editing are summarized in the sections below:

### A. Unquantifiable or Missing Data

Pomalidomide concentration values below the quantifiable limit (BLQ) were excluded from the PPK analysis dataset. Pomalidomide concentrations in the PPK analysis dataset were excluded from the analysis if missing data pertaining to the dose or covariate values could not be appropriately imputed.

Whenever possible, the actual elapsed time of sampling relative to either a prior dose or the first administered dose (as appropriate for the study design) was used as the time variable.

Actual dose time was missed for all predose sparse PK samples collected on Cycle 1 Day 8 and Cycle 1 Day 15 in Study 5, and therefore actual time of blood sample collection relative to previous dose for these predose PK data were imputed as 24 hours after the current dose. All sparse PK samples collected on Cycle 1 Day 22 in Study 5 were excluded from PPK analysis due to inaccurate dosing data/time records.

The baseline value of a continuous covariate was defined as the measurement obtained prior to the first dose, with the value on Cycle 1 Day 1 as the first choice. If multiple values were obtained on Day 1, the first value was used as the baseline unless the repeated values suggested otherwise. For subject who were missing covariate values at baseline (Cycle 1 Day 1) but had a screening value, the screening values were used for the imputation. A covariate was imputed based on median (continuous) or mode (categorical) covariates if values were missing for less than 10% of the patients. A continuous or categorical covariate was excluded from the analysis if values were missing for more than 10% of the subjects

### B. Unquantifiable or Missing Data

Data visualization was performed to search for patterns and extreme values that may have caused significant bias during the analysis. Aberrant values were identified prior to the initiation of the analyses by distributional checks, as well as in initial modeling using conditional weighted residuals (CWRES). The outliers were considered influential if their exclusion from the analysis resulted in a change of greater than 10% in base model parameters. In addition, the aberrancy of a PK observation in relation to the temporal nature of a subject's profile was also taken into account in determining whether the observation was an outlier.

If the outliers were not influential, these values were included in the development of the full model. If the outliers were influential, these observations were not included in further model development.

### 7.1.2.3.4 Population Pharmacokinetic Modeling

### 7.1.2.3.4.1 Structure Model Characterization

Concentration data were transformed by natural logarithm (Ln) prior to PPK modeling. The concentration-time profiles were visually inspected for mono-, or bi-exponential decline. Based on visual inspection, the starting point was a one-compartment structural PK model with the first-order elimination and distribution. Various alternative models were tested to identify the structural model that best described the data. A two-compartment structure PK model was selected based on the objective function value (OFV) using the log-likelihood ratio test and the goodness-of-fit criteria. The final structure PK model was parameterized in terms of:
Absorption lag time in healthy subjects (Alag1_{HNS})
First order absorption rate constant (ka)
Apparent volume of distribution for the central compartment in healthy subjects (V2/F)
Apparent volume of distribution for the peripheral compartment in healthy subjects (V3/F)
Apparent inter-compartmental clearance between the central compartment and the peripheral compartment in healthy subjects (Q/F)
Apparent clearance in healthy subjects (CL/F)
Absorption lag time in MM patients (Alag1_{MM})
Ratio of apparent clearance between MM patients and healthy subjects (CL/F_{MM patient/HNS})
Ratio of apparent volume of distribution for the peripheral compartment between MM patients and healthy subjects (V3/F_{MMpatient/HNS})
Ratio of inter-compartmental clearance between MM patients and healthy subjects (Q/F_{MM patient/HNS})
Ratio of volume of distribution for the central compartment between MM patients and healthy subjects (V2/F_{MM patient}/_{HNS})

Unexplained interindividual variability (IIV) in a PK parameter was modeled as follows:

Pₜ=P·eⁿⁱ

where P was the typical value of the parameter in the population, Pi was the value of the parameter for the ith individual, and ni was a random interindividual effect in the parameter for the ith subject with a mean of zero and variance ω2 (i.e., η˜N[0, ω²]). It was assumed in this model that the Pi values were log-normally distributed. Interindividual variability in P was estimated as (exp(ω²)−1)^{0.5}, which is an approximation of the coefficient of variation of P for a log-normally distributed quantity. Other models were considered if indicated by the data.

Intra-individual or residual variability (RV) was modeled as follows:

Ln(C_{if})=Ln(C_{mif})+Cᵢⱼ

where Cₘᵢⱼ was the model-predicted jth concentration in the ith subject, Cᵢⱼ was the observed jth concentration in the ith subject, and εij was the random residual effect for the jth concentration in the ith subject with mean zero and variance of σ2. Residual variability in Cₘᵢⱼ was estimated as (exp(σ²)−1)^{0.5}, which is the coefficient of variation of Cₘᵢⱼ for a log-normally distributed quantity. The value of σ² may vary between individuals. Therefore, the assumption of a constant σ² for all individuals may result in biased variable estimates. To reduce this possible bias, two different values for σ² were supposed (σ1² for healthy normal subjects, and σ2² for patients with multiple myeloma).

Selection of the structural model was based on:
A. Diagnostic plots
   Observed values (DV) versus population predicted values (PRED) and individual predicted values (IPRED)
   CWRES versus PRED concentrations
   CWRES versus time
   Goodness-of-fit for individual subject profiles
B. Statistical criteria: the log-likelihood difference between rival models (i.e., change in the minimum objective function value [OFV]). A difference in OFV between two nested models is approximately χ2-distributed. A difference in OFV>3.84, 6.63, and 10.83 (one degree of freedom) is thus significant at the 5%, 1%₅ and 0.1% level, respectively.
c. Other goodness-of-fit criteria: inter-individual variability (IIV), residual variability (RV), variance, and the precision of parameter estimates.

### 7.1.2.3.4.2 Parameter Shrinkage

The extent of shrinkage of empiric Bayesian parameter estimates towards the population mean of the parameter (ηₛₕ) were calculated as:

$${η}_{sh} = 1 - \frac{SD ( {\hat{η}}_{sh} )}{ω}$$

where SD was the standard deviation of the interindividual variability (ω) for a parameter

The extent of shrinkage of individual predicted concentrations towards the population mean concentration (εₛₕ) was calculated as:

$${ɛ}_{sh} = 1 - SD ( \frac{{DV}_{ij} - {IPRED}_{ij}}{σ} )$$

where SD was the standard deviation of the residueal variability (σ), Dvᵢⱼ was the ith observed drug concentration in the jth individual, and IPREDᵢⱼ was the ith predicted drug concentration in the jth individual.

### 7.1.2.3.4.3 Graphic Analysis

Covariate model development was performed through a combination of data visualization (graphing and fitting using locally weighted regression) in S-PLUS and nonlinear mixed effect modeling. Pair plots (scatterplot matrices) were used to examine relationships between PK parameters (CL/F and V2/F) and continuous covariates. Boxplots of empirical individual Bayesian estimates, sorted by categorical/binary covariates, were also used to examine potential relationship between parameters and such covariates. The results of the graphical analysis were used to inform the covariates that were considered for testing in the NONMEM analysis for the development of a predictive PPK model for pomalidomide.

### 7.1.2.3.4.4 Covariate Analysis

The following variables were explored as covariates for their potential to influence pomalidomide PK parameters:
Demographic factors: age, body weight, body surface area, sex, and race
Hepatic function markers: total bilirubin, albumin, AST, or other markers as appropriate
Renal function marker: creatinine clearance estimated by Cockcroft-Gault formula
Status of health (healthy subjects versus patients)

Empirical Bayesian estimates of the parameters were generated for each subject. Shrinkage was examined to determine whether the PK parameter estimates were reliable. Data visualization was used to examine the relationship between intrinsic or extrinsic factors and subject-level PK parameters. Initial selection of covariates was guided by graphic inspection and biological plausibility. Potential covariates were tested further in NONMEM.

The stepwise covariate model (SCM) building tool of PsN was used for the development of pomalidomide covariate model, which implemented forward selection and backward elimination of covariates to pomalidomide PPK model. In short, one model for each relevant parameter-covariate relationship is prepared and tested in a univariate manner.

In the first step, the model that gives the best fit of the data according to some criteria is retained and taken forward to the next step. In the following steps all remaining parameter-covariate combinations are tested until no more covariates meet the criteria for being included into the model. The forward selection is followed by backward elimination which proceeds as the forward selection but reversely, using stricter criteria for model improvement.

There is a fixed set of PK parameter-covariate relations defined in SCM, the predefined shapes for the parameter-covariate relations for continuous covariates for pomalidomide covariate model development include:

linear equation:

P=θ·(1+θ_{cov}*(COVᵢ−COVₘ))

and power equation:

$$P = θ * {( \frac{{Cov}_{i}}{{Cov}_{m}} )}^{{θ}_{COV}}$$

where P was the typical value of a PK parameter in the population after adjusting for values of covariates of individual subjects, θ was the typical value of the PK parameter, θ_{cov} was the coefficient for the effect of the covariate, COVᵢ was the covariate value for individual subjects, and COVₘ was the median value of the covariates in the study population. Categorical covariates were included in the pomalidomide covariate model development is:

P=θ*(1+θ_{cov}*Z_{ind,k})

where Z_{ind,k} is an indicator variable representing one from of a binary covariate and θ_{cov} was the coefficient for the effect of the covariate.

A three-stage approach for the selection of covariates with NONMEM was followed:
1. In the first step (univariate analysis), covariates identified by the graphic analysis were introduced into the structural model one at a time. For a covariate to be claimed to have no effect, a univariate analysis of this covariate was also preformed regardless of results from the graphic analysis, and the 90% or 95% confidence interval (CI) for the estimated effect was provided. The effect of a covariate was subsequently tested in the forward selection if its inclusion in the PPK model resulted in a decrease in OFV greater than 3.84 (X²−test, degree of freedom=1, p<0.05) in the univariate analysis.
2. In the second step (forward selection), the covariate that had the highest significance in the univariate analysis was included first, and the other significant covariates from the first step were included in the rank order of their significance. The covariate effect was included in the PPK model if a decrease in OFV greater than 6.63 (X²−test, degree of freedom=1, p <0.01) was observed. This process was repeated until there were no significant covariates remaining The process led to the full multivariate mode.
3. In the third step (backward elimination), covariates were removed from the full model obtained from the second step, one at a time until there were no further insignificant covariates in the model. If the exclusion of a covariate resulted in an increase in OFV of less than 10.83 (X²−test, degree of freedom=1, p>0.005), the covariate was excluded from the model. A conservative p-value of 0.001 was selected to avoid the inclusion of weak and clinically non-relevant effects. Using this approach, a final model was constructed.

The 90% or 95% CI for a covariate parameter estimate not including zero (for continuous covariate) or 1 (for categorical covariate), a reduction in unexplained interindividual variability, and clinical significance were also considered for retention of a possible explanatory covariate.

### 7.1.2.3.4.5 Reliability of Parameter Estimates

NONMEM covariance step was not implemented during the modeling process and with each PPK model developed. Stability of the final PK parameter estimates and the 95% CI for the parameters were evaluated using the nonparametric bootstrap approach. With this approach, 500 datasets of size equal to the original dataset were generated by random resampling with replacement from the original dataset. The final model was fit to each of the 500 bootstrap datasets and all the model parameters were estimated for each dataset. The median and nonparametric 95% CI (2.5-97.5 percentiles) of the 500 estimates were calculated for each parameter.

### 7.1.2.3.4.6 Predictive Performance

Model evaluation was performed using visual predictive check (VPC) that provides an evaluation of model assumptions and population parameter estimates by comparing model predictions with observations. The ability of the final PPK model to describe the observed concentration data was evaluated by simulating 500 datasets having the same doses, dosing schedules and sampling times as the original dataset and by performing VPCs. Binning of observations around logical postdose time points of interest was done to ensure sufficient density of observations. The 5th, 50th and 95th prediction percentiles of the pomalidomide concentrations at each binned time point were computed for each simulated trial. Thereafter, the nonparametric 95% CI of the 5th, 50th and 95th prediction percentiles at each binned time point were calculated for the 500 simulated trials. The data were displayed graphically and overlaid with the corresponding percentiles of the observed data.

### 7.1.2.3.4.7 Generation of Subject-Specific Exposure Measures

Empiric individual Bayesian estimates of PK parameters were generated using the final PPK model. With these individual parameter estimates, the concentration-time profile was simulated and appropriate measures of pomalidomide exposure at steady state were computed for each subject. Potential exposure measures included the following:
AUC₂₄: area under the concentration-time curve (from time zero to 24 hours after dosing) at steady state
Cₘₐₓ: maximum concentration at steady state

These exposure measures were used, as appropriate, in exposure-response analyses.

### 7.1.3 Results

### 7.1.3.1 Summary of Analysis Dataset

The final dataset used for PPK modelling is summarized in Table 2 below. A total of 236 subjects with 3909 evaluable pomalidomide concentration records were included in the final population PPK analysis dataset. These data were obtained from 6 studies (Table 2). One subject from study 5 was excluded from the PPK analysis due to missing Cycle 1 Day 1 dosing time. Approximately 15% of the concentration records were excluded from PPK analysis for the reasons below:
Concentrations below the limit of quantification (66.57%)
Concentrations above the limit of quantification from Cycle 1 Day 1 predose samples (0.14%)
Concentrations missing dose time (11.58%)
Wrong actual PK sampling time (0.14%)
Concentrations missing collection time (6.8%)
Protocol deviation (PK data collected from Cycle 1 Day 22 in study 5) (14.76%).

TABLE 2
Subjects and Concentration Records Included

| | Number of | | | Number of | | |
|---|---|---|---|---|---|---|
| | Subjects | | | Concentration Records | | |
| | | Excluded | Included | | Excluded | Included |
| Study | Total | (%) | (%) | Total | (%) | (%) |
| 1 | 24 | 0 (0%) | 24 (100%) | 693 | 88 (13%) | 605 (87%) |
| 2 | 72 | 0 (0%) | 72 (100%) | 2256 | 230 (10%) | 2026 (90%) |
| 3 | 28 | 0 (0%) | 28 (100%) | 802 | 129 (16%) | 673 (84%) |
| 4 | 14 | 0 (0%) | 14 (100%) | 208 | 17 (8%) | 191 (92%) |
| 5 | 80 | 4 (5%) | 76 (95%) | 505 | 222 (44%) | 283 (56%) |
| 6 | 22 | 0 (0%) | 22 (100%) | 136 | 5 (4%) | 131 (96%) |
| Total | 240 | 4 (2%) | 236 (98%) | 4600 | 691 (15%) | 3909 (85%) |

Demographic and baseline characteristics are summarized for the PPK analysis population in Table 3. The subjects were primarily White (78%), not Hispanic or Latino (65.7%), and male (74.2%). They had a median (range) age of 53.0 (19.0, 83.0) years. The median (range) creatinine clearance, a marker associated with renal function, was 100.4 (20.8, 188.2) mL/min.

TABLE 3
Demographic and Baseline Characteristics of Analysis Population

| Variables | | Statistics | Values | |
|---|---|---|---|---|
| Sex | Male | Number (%) | 175 | (74.2%) |
| | Female | Number (%) | 61 | (25.8%) |
| Race | Asian | Number (%) | 2 | (0.8%) |
| | Black or African | Number (%) | 47 | (19.9%) |
| | American | | | |
| | Native Hawaiian or | Number (%) | 1 | (0.4%) |
| | Other Pacific | | | |
| | Islander | | | |
| | Other | Number (%) | 2 | (0.8%) |
| | White | Number (%) | 184 | (78%) |
| Ethnicity | Hispanic or Latino | Number (%) | 53 | (22.5%) |
| | Not Hispanic or | Number (%) | 155 | (65.7%) |
| | Latino | | | |
| | Unknown | Number (%) | 28 | (11.9%) |

| | | | |
|---|---|---|---|
| Age (year) | Median (range) | 53.0 | (19.0, 83.0) |
| Body weight (kg) | Median (range) | 78.1 | (44.4, 127.0) |
| Height (cm) | Median (range) | 171.5 | (142.0, 191.3) |
| Body mass index (kg/m²) | Median (range) | 26.5 | (16.4, 48.3) |
| Albumin (g/dL) | Median (range) | 40.0 | (17.0, 52.0) |
| Total bilirubin (μM) | Median (range) | 9.2 | (1.9, 52.3) |
| Total protein (g/L) | Median (range) | 75.0 | (56.0, 148.0) |
| Asparate aminotransferase (U/L) | Median (range) | 22.0 | (9.0, 73.0) |
| Alkaline phosphatase (U/L) | Median (range) | 60.6 | (25.0, 255.0) |
| Serum creatinine (μmol/L) | Median (range) | 85.7 | (47.7, 332.0) |
| Creatinine clearance (mL/min) | Median (range) | 100.4 | (20.8, 188.2) |

The concentration versus time profiles for individual subjects showed that most subjects had evaluable PK profiles for an appropriate PPK analysis.

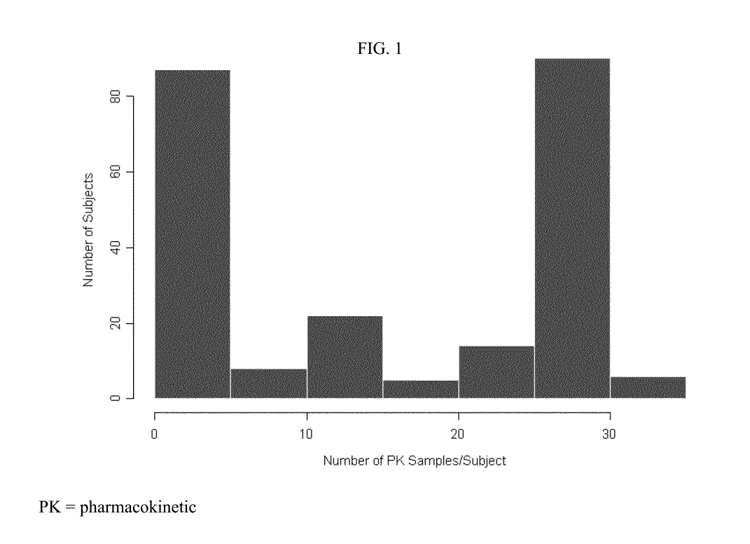

FIG. 1 showed that 35 samples, at most, were obtained per subject in the PPK dataset and a majority of subjects supplied less than 5 samples or between 25 to 30 samples indicating that the number of subjects who supplied intensive and sparse PK samples was well balanced in the current PPK dataset.

### 7.1.3.2 Structural Pharmacokinetic Model Characterization

Various structure models were tested to identify the appropriate structure PPK model for pomalidomide and the major results of the model build up are summarized in Table 4 below:

TABLE 4
Major Results of Structure Population Pharmacokinetic Model Build Up

| Model Description | OFV | Residual Error (σ²) |
|---|---|---|
| 1 compartment model | −2054.34 | 0.149 |
| 2 compartment model | −2536.75 | 0.1240 |
| 2 compartment model with a lag time | −3160.048 | 0.10 |
| Fix ETA(Q/F) = 0 | −3160.047 | 0.10 |
| Different residual error for healthy | −4528.96 | σ²(HNSs) = 0.04 |
| subjects and MM patients | | σ²_{(MM patients)} = 0.282 |
| Different CL/F for healthy subjects | −4368.217 | σ²(HNSs) = 0.0406 |
| and MM patients | | σ²(MM patients) = |
| | | 0.283 |
| Different lag time for healthy | −4663.919 | σ²(HNSs) = 0.0399 |
| subjects and MM patients | | σ²(MM patients) = |
| | | 0.253 |
| Different V₃/F for healthy subjects | −4736.318 | σ²(HNSs) = 0.0404 |
| and MM patients | | σ²(MM patients) = |
| | | 0.239 |
| Different Q/F for healthy subjects | −4811.301 | σ²(HNSs) = 0.0404 |
| and MM patients | | σ²(MM patients) = |
| | | 0.239 |
| Different V₂/F for healthy subjects | −4823.449 | σ²(HNSs) = 0.04 |
| and MM patients | | σ²(MM patients) = |
| | | 0.240 |
| Set correlation for ETA(V₂/F) and | −4872.452 | σ²(HNSs) = 0.0399 |
| ETA(CL/F) | | σ²(MM patients) = |
| | | 0.241 |
| Fix ETA(V₃/F) = 0 | −4872.172 | σ²(HNSs) = 0.04 |
| | | σ²(MM patients) = |
| | | 0.241 |

ETA=inter-individual variability; CL/F=apparent clearance; HNS=healthy normal subject; MM=multiple myeloma; OFV=objective function value; Q/F=inter-compartmental clearance; V2/F=apparent central volume of distribution; V3/F=apparent peripheral volume of distribution; σ2=residual error.

To identify the structural model, a one-compartment PK model was compared with a two compartment PK model. The two-compartment model (OFV: −2536) with first order oral input was preferred over the one-compartment model with first order oral input (OFV: −2054). In addition, incorporating a lag time improved the model fitting by significantly decreasing OFV from −2536 to −3160.

Considering inherently better quality of PK data collected from well-controlled studies in healthy subjects compared to that collected from studies in patients, different residual error model (OFV: −4528) was tested and preferred over the model with same residual error model (OFV: 3160).

A visual examination of the dose normalized concentration versus time profiles from healthy normal subjects and MM patients showed longer terminal phase in MM patients than in healthy normal subjects indicating possibly deeper tissue/organ distribution of pomalidomide in MM patients. Therefore a two-compartment model with different Q/F and V3/F between MM patients and healthy subjects (OFV: −4823) was tested and preferred over the two-compartment model with same Q/F and V3/F between MM patients and healthy subjects (OFV: −4528).

According to goodness-of-fit and statistical criteria, a two-compartment model with first order absorption rate constant incorporating a lag time, and different lag time, CL/F, Q/F, V3/F, V2/F and error model between MM patients and healthy subjects adequately described pomalidomide PK in both healthy subjects and MM patients, and was selected as the final structural PPK model.

Overall, observed concentrations of pomalidomide were well-fitted with the structural PPK model and error model.

The PPK parameters from the structural PPK model are presented in Table 5 below. The structural model parameters were precisely estimated with the base model as observed from the narrow 95% bootstrap confidence intervals. The IIV associated with the PK parameters in the base model were estimated as log-normally distributed with a non-zero covariance in the IIV on CL/F and V2/F. The estimated IIV and associated covariance were reasonably precise. The data did support the estimation of IIV in Q/F and V3/F. The V2/F and CL/F were estimated with a reasonably acceptable shrinkage (20.4% and 7.75%, respectively).

TABLE 5
Pharmacokinetic Parameter Estimates from the Base Model

| | | Bootstrap | | Shrinkage |
|---|---|---|---|---|
| | Estimate | Estimate | 95% Bootstrap CI^{a} | (%) |
| ka (hr⁻¹) | 1.23 | 1.23 | (1.05, 1.44) | 14.4 |
| V₂/F (L) | 58.9 | 58.91 | (56.78, 61.54) | 20.4 |
| V₃/F (L) | 8.49 | 8.53 | (7.51, 9.51) | |
| Q/F (L/hr) | 1.01 | 1.01 | (0.77, 1.29) | |
| CL/F (L/hr) | 8.52 | 8.51 | (8.04, 8.98) | 7.75 |
| ALAG1 (hr) | 0.385 | 0.38 | (0.37, 0.40) | |
| CL/F_{(patient/HNS)} | 0.81 | 0.81 | (0.73, 0.91) | |
| ALAG1ₚₐₜᵢₑₙₜₛ | 0.206 | 0.21 | (0.18, 0.23) | |
| (hr) | | | | |
| V₃/F₍ₚₐₜᵢₑₙₜ/_{HNS)} | 8.51 | 8.42 | (5.89, 11.95) | |
| Q/F₍ₚₐₜᵢₑₙₜ/_{HNS)} | 3.74 | 3.72 | (2.46, 5.47) | |
| V₂/F₍ₚₐₜᵢₑₙₜ/_{HNS)} | 1.19 | 1.19 | (1.06, 1.32) | |

Inter-Individual Variability (IIV)

| | | | |
|---|---|---|---|
| ω² (ka) | 1.02 | 0.998 | (0.674, 1.414) |
| ω² (V₂/F) | 0.0488 | 0.049 | (0.032, 0.070) |
| ω² (V₂/F): ω² | 0.0680 | 0.067 | (0.042, 0.098) |
| (CL/F) | | | |
| ω² (CL/F) | 0.178 | 0.174 | (0.133, 0.232) |

Residual Variability

| | | | | |
|---|---|---|---|---|
| δ^{2 12/21} (HNS) | 0.040 | 0.0398 | (0.0330, 0.0470) | 5.24 |
| δ² (Patients) | 0.241 | 0.237 | (0.195, 0.295) | 7.93 |

ALAG1 = lag time;
CI = confident interval;
CL/F = apparent clearance;
CL/F_{(patient/HNS)} = ratios of apparent clearance between MM patients and healthy subjects;
IIV = inter-individual variability;
ka = absorption rate constant;
Q/F = apparent inter-compartmental clearance between the central compartment and the peripheral compartment;
Q/F_{(patient/HNS)} = ratios of apparent inter-compartmental clearance between the central compartment and the peripheral compartment between MM patients and healthy subjects;
V₂/F = apparent volume of distribution for the central compartment;
V₂/F_{(patient/HNS)} = ratios of apparent volume of distribution for the central compartment between MM patients and healthy subjects;
V₃/F = apparent volume of distribution for the peripheral compartment;
V₃/F_{(patient/HNS)} = ratios of apparent volume of distribution for the peripheral compartment between MM patients and healthy subjects;
^{a}Bootstrap confidence interval values are taken from bootstrap calculation (498 successful out of a total of 500 bootstrap replicates)

### 7.1.3.3 Covariate Analysis

### 7.1.3.3.1 Exploratory Graphical Analysis

Since the shrinkage associated with structural model parameters for V2/F and CL/F were acceptable, individual empirical Bayesian estimates were generated for the examination of the relationship between the structural model parameters (V2/F and CL/F) and potential covariates. The CL/F appeared to be positively related to body weight, ALB and CLCR values, and appeared to be negatively related to age.

The V2/F appeared to be positively related to age, body weight, and TPT levels, and appeared to be negatively related to ALB and BIL values.

When categorical covariates were considered, female subjects appeared to have significantly lower CL/F as compared to male subjects, and in addition, Hispanic or Latino and non-Hispanic subjects appeared to have significantly different CL/F values. There was no observable relationship between V₂/F and sex, race or ethnicity.

For the categorical covariate of race, the PPK dataset contained majority of White population (N=180). There are only 47 Black or African Americans, 1 native Hawaiian or other Pacific Islander population, 2 Asians and 2 Others. Therefore, all non-White populations were grouped as one population in the following covariate analysis.

For the categorical covariate of ethnicity, the PPK dataset contained majority of Not Hispanic or Latino (N=151) population. There are only 53 Hispanic or Latino and 28 Unknowns. Therefore, Hispanic or Latino and unknown were grouped as one ethnic group (Hispanic or unknown) in the following covariate analysis.

### 7.1.3.3.2 Covariate Model Development

Even though only a few covariates were identified in the exploratory graphic analysis, all proposed covariates were included in the covariate model development using the stepwise covariate model (SCM) building tool of PsN.

The output of stepwise covariate model building log file showed that inclusion of body weight and TPT into V2/F and inclusion of sex into CL/F in the forward selection step significantly improved the model fitting. Both linear and power equations were tested for the V2/F versus body weight and TPT relationship. A linear equation between V2/F and TPT values, and a power equation between V2/F and body weight statistically significantly improved the model fitting by a decrease of OFV from −4872 to −4944.

Although body weight, ALB levels and CLCR levels appeared to be positively correlated with CL/F, age appeared to be negatively correlated with CL/F, age appeared to be positively correlated with V2/F, and ALB levels and BIL levels appeared to be negatively correlated with V2/F in the graphic analyses, these trends did not reach statistical significance in forward selection step.

The final model was identified through a backward elimination process. No covariates identified from forward selection step were removed from the full model. A covariate was dropped if its removal from the full model resulted in an increase of <7.879 in OFV (p>0.005).

### 7.1.3.3.3 Final Model

The PK parameters from the final PPK model for pomalidomide are presented in Table 6 below.

TABLE 6
Pharmacokinetic Parameter Estimates from the Final Model

| | | Bootstrap | 95% | Shrinkage |
|---|---|---|---|---|
| | Estimate | Estimate | Bootstrap CI^{a} | (%) |
| KA (hr⁻¹) | 1.25 | 1.25 | (1.07, 1.47) | 14.8 |
| V₂/F (L) | 58.3 | 58.25 | (55.79, 60.88) | 19.9 |
| V₃/F (L) | 8.45 | 8.48 | (7.47, 9.40) | |
| Q/F (L/hr) | 1.01 | 1.00 | (0.75, 1.28) | |
| CL/F (L/hr) | 8.52 | 8.51 | (8.04, 8.99) | 7.89 |
| ALAG1 (hr) | 0.385 | 0.38 | (0.37, 0.40) | |
| CL/F_{(patient/HNS)} | 0.913 | 0.911 | (0.798, 1.056) | |
| ALAG1ₚₐₜᵢₑₙₜₛ (hr) | 0.206 | 0.207 | (0.177, 0.231) | |
| V₃/F₍ₚₐₜᵢₑₙₜ/_{HNS)} | 8.46 | 8.38 | (5.92, 11.80) | |
| Q/F₍ₚₐₜᵢₑₙₜ/_{HNS)} | 3.71 | 3.68 | (2.50, 5.35) | |
| V₂/F₍ₚₐₜᵢₑₙₜ/_{HNS)} | 1.20 | 1.19 | (1.07, 1.37) | |
| TPT on V₂/F | 0.00609 | 0.00602 | (0.0024, 0.0103) | |
| WT on V₂/F | 0.686 | 0.685 | (0.497, 0.862) | |
| Sex on CL/F | −0.234 | −0.232 | (−0.369, −0.077) | |

Inter-Individual Variability (IIV)

| | | | |
|---|---|---|---|
| ω² (ka) | 0.976 | 0.961 | (0.652, 1.388) |
| ω² (V₂/F) | 0.0352 | 0.0349 | (0.0240, 0.050) |
| ω² (V₂/F): ω² | 0.0599 | 0.0589 | (0.0395, 0.0841) |
| (CL/F) | | | |
| ω² (CL/F) | 0.168 | 0.164 | (0.127, 0.213) |

Residual Variability

| | | | | |
|---|---|---|---|---|
| δ² (HNS) | 0.04 | 0.04 | (0.033, 0.047) | 5.09 |
| δ² (Patients) | 0.240 | 0.235 | (0.192, 0.295) | 7.65 |

ALAG1 = lag time;
CI = confident interval;
CL/F = apparent clearance;
CL/F_{(patient/HNS)} = ratios of apparent clearance between MM patients and healthy subjects;
IIV = inter-individual variability;
ka = absorption rate constant;
Q/F = apparent inter-compartmental clearance between the central compartment and the peripheral compartment;
Q/F_{(patient/HNS)} = ratios of apparent inter-compartmental clearance between the central compartment and the peripheral compartment between MM patients and healthy subjects;
V₂/F = apparent volume of distribution for the central compartment;
V₂/F(_{patient/HNS)} = ratios of apparent volume of distribution for the central compartment between MM patients and healthy subjects;
V₃/F = apparent volume of distribution for the peripheral compartment;
V₃/F_{(patient/HNS)} = ratios of apparent volume of distribution for the peripheral compartment between MM patients and healthy subjects.
^{a}Bootstrap confidence interval values are taken from bootstrap calculation (483 successful out of a total of 500 bootstrap replicates).

In the final PPK model, the IIV was determined for ka, V₂/F and CL/F. Inclusion of IIV for the other PK parameters did not improve the fit, indicating that the data did not contain sufficient information to estimate these variables. Most of the PK parameters for the final model were estimated with good precision (relatively narrow 95% CI from 500 bootstrap runs).

Inclusion of sex as the covariate of CL/F and body weight and TPT as the covariates of V₂/F was found to improve the goodness-of-fit of the model in a statistically significant manner. The final covariate model at the population level was described as follows:

$$\frac{CL}{{P}_{TV}} = \begin{matrix}0.45 & for male subjects \\ 8.45 * ( 1 - 0.234 ) & for female subjects\end{matrix}$$

$$and$$

$$\frac{V 2}{{F}_{TV}} = 58.3 * {( \frac{WT}{78.3} )}^{0.686} * ( 1 + 0.00609 * ( TPT - 73.0 ) )$$

Typical value of pomalidomide CL/F was 8.45 for male subjects as reference, and typical values of pomalidomide V₂/F was 58.3 at a median body weight of 78.3 kg, and at a median TPT level of 73 g/L. The final model suggests that female subjects showed 23.4% lower apparent clearance as compared to male subjects, and V₂/F increases with increasing body weight or TPT levels. Despite the statistical significance of the effect of sex on CL/F, and the effect of body weight and TPT on V₂/F, the contribution of sex to the IIV of CL/F and the contribution of body weight and TPT levels were marginal, reducing the IIV from 44.1% in the base model to 42.77% in the final model, and from 22.3% in the base model to 18.9% in the final model for CL/F and V₂/F, respectively. Therefore, none of these covariates (sex on CL/F, body weight and TPT on V₂/F) appears to be clinically relevant.

The comparison of goodness-of-fit plots for base and final model showed a general reduction of bias especially at high concentrations. A comparison of the observed versus predicted concentration plot for the base and final models showed that the bias in the population prediction of the high concentrations observed with the base model was improved with the final model. This would suggest that the covariates in the final model might be the relevant covariates for explaining the variability in the pomalidomide PK, given the data, thereby yielding good individual predictions.

The difference in unexplained variability in CL/F when plotted against sex category observed with the base model was eliminated in the final model.

The observed time courses of pomalidomide concentrations were well predicted by the final PPK model across all dose levels (0.5 to 10 mg), with the observed concentrations well overlapped with the model-predicted concentrations at each time point. This indicated that the final PPK model adequately characterized pomalidomide PK without observable bias on different dose levels.

### 7.1.3.3.4 Model Evaluation

The PPK model available after completing the covariate analysis steps was subjected to bootstrap resampling stability testing to establish the robustness of the model. A total of 500 bootstrap runs were performed using the final PPK model, with 483 runs (96.6%) successfully minimized. As shown in Table 6, the median values of the parameters obtained from bootstrap replications were similar to the original NONMEM estimates. The relative difference between the final model estimate and the bootstrap median was 15% for the fixed effect parameters and 4% for the random-effect parameters. These results suggested that the final model is robust and stable.

Approximately 90% of the observed concentration data were well contained within the 90% prediction intervals. The 5th, 50th and 95th percentiles of the observed concentration data at each time point were generally contained within the respective 95% CI. There was a good agreement in the time course and central tendency between distributions of observed and simulated data, with no obvious bias. Overall, the estimated IIV adequately described the observed variability in pomalidomide concentrations.age.

Taken together, pomalidomide concentrations in the Log range of 0.006 to 5.19 (ie, 1.0 to 179 ng/mL) were well characterized by the final PPK model.

These exposure measures were used, as appropriate, in exposure-response analyses.

### 7.1.4 CONCLUSION

The pomalidomide PPK model provided an adequate description of pomalidomide concentration-time data from both healthy subjects and MM patients. Two hundred and thirty-six subjects provided 3909 concentrations that were used in the development of the integrated PPK model for pomalidomide. Pomalidomide concentration-time data was well characterized by the structural PK model that consists of a two-compartment with first order absorption incorporating a lag time and first-order elimination.

Goodness-of-fit plots indicated that the present PPK model was fitted well with the observed data. The final PPK model was evaluated with bootstrap re-sampling procedure. The bootstrap parameters were within ±10% of the final PPK parameters obtained from the original dataset, and more than 95% of 500 re-sampling dataset converged which demonstrated robust stability and adequate predictive performance of the final PPK model. The predicted check (VPC) showed that the linear two-compartment model adequately characterized the data. Thus, the model was deemed appropriate for its intended purposes of the characterization of pomalidomide PPK and the generation of exposure metrics for use in exposure-response analyses.

The present analysis indicated that the drug is rapidly absorbed with a model predicted absorption rate constant of 1.25 h⁻¹ which was estimated with good precision since there were a sufficient number of observations in the absorption phase from healthy subjects. Pomalidomide exhibits linear, time independent PK. Following oral administration, pomalidomide undergoes biphasic disposition. Healthy subjects and MM patients showed comparable apparent clearance (CL/F) (8.45 and 7.7 L/h, respectively) and apparent central volume of distribution (V₂/F) (58.3 and 69.9 L, respectively).

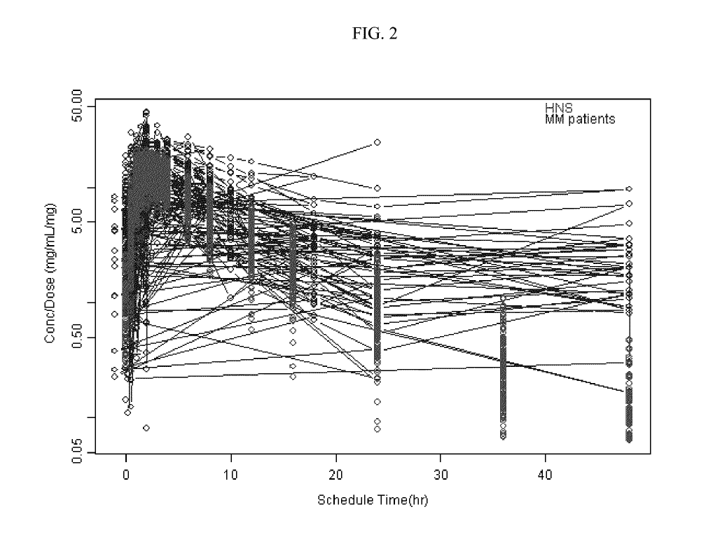

A visual examination of the dose normalized concentration versus time profiles from healthy subjects and MM patients (FIG. 2Error! Reference source not found.) showed longer lingering of plasma concentrations at the terminal phase in MM patients than in healthy subjects indicating possibly deeper tissue/organ distribution of pomalidomide in patients. Therefore a two-compartment model with different apparent inter-compartment clearance (Q/F) and apparent peripheral volume of distribution (V₃/F) between MM patients and healthy subjects was tested and preferred over the two compartment model with same Q/F and V₃/F between MM patients and healthy subjects. Based on the current analysis, V₃/F and Q/F in MM patients were found to be higher than that in healthy subjects (approximately 8-fold and 3.7-fold, respectively) indicating an effect of disease on the disposition of pomalidomide which is consistent with pomalidomide action on malignant plasma cells.

In the covariate analysis, the most influential covariates in the disposition of pomalidomide were gender, body weight and total protein (TPT) levels. The effect of gender on CL/F and body weight and TPT levels on V₂/F were found statistically significant in improving the model fitting.

Although renal function marker (CrCL) appeared to be positively correlated with CL/F in the graphic analyses (correlation coefficient=0.2042), this correlation did not reach statistical significance in the following covariate model assessment indicating that the kidney does not contribute to the pomalidomide elimination in vivo. In the current PPK dataset, there is an adequate distribution of MM patients with different renal function (40 subjects with normal renal function, 54 subjects with mildly impaired renal function, 40 subjects with moderately impaired renal function, and 3 subjects with severely impaired renal function), suggesting the non-significant association between renal function marker (CLcr) and apparent clearance (CL/F) is applicable for a wide range of CLcr observed (i.e. 20.8 to 188.2 mL/min).

Although one of the hepatic function markers (ALB level) appeared to be positively correlated with CL/F (correlation coefficient=0.185) in the graphic analyses, this correlation did not reach statistical significance in the covariate analysis. Another hepatic function marker (AST level) did not show correlation with CL/F. However, all subjects included in this analysis showed normal ALB levels (a range from 17 to 52 g/dL), and the number of patients with elevated AST findings in this analysis was small (2 patients with AST 1.5-fold higher than ULN). Therefore, the relationship between the hepatic function and the CL/F should be interpreted with caution.

Body weight (a range from 44.4 to 127 kg) appeared to be positively correlated with CL/F (correlation coefficient=0.188) and age (a range from 19 to 83 years) appeared to be negatively correlated with CL/F (correlation coefficient=−0.202) in the graphic analyses. However, inclusion of these covariates in the PPK model yielded a statistically non-significant association.

Race (White versus non-white) was found not to have any effect on pomalidomide PK. The PPK dataset contained majority of Whites, and only 2 Asians, 1 native Hawaiian or Other Pacific and 47 African Americans. Thus, the lack of effect of race on pomalidomide PK is only limited to the White and non-White population that consisted of 184 and 52 subjects, respectively, in the PPK dataset.

The present analysis indicated that the systemic clearance of the drug was only significantly correlated to gender according to the criteria defined for covariate analysis. Sex-related pharmacokinetic disparities have been reported for other drugs. There are many potential reasons for gender differences in pomalidomide pharmacokinetics, such as differences in gastric pH, which is higher in females, lower hepatic blood flow and consequently lower hepatic metabolic capacity, which could partly explain the findings. The resulting differences in drug exposures between female and male subjects were small (below 30%) relative to the observed overall variability in the PK of pomalidomide.

Body weight was a significant factor influencing pomalidomide central volume of distribution. Large volume of distribution of pomalidomide suggests that pomalidomide may be distributed by diffusion into the extracellular fluids, the volume of which increases with body weight; thus, the estimated increases in the central volume of distribution of pomalidomide with increased body weight are consistent with the physiological effects of weight.

Total protein level (TPT) (a range from 56 to 148 g/L) was another significant factor influencing pomalidomide central volume of distribution. MM disease is associated with an elevated total protein levels and higher disease stage is also correlated with higher serum protein levels. Higher total protein level may correlates with higher disease stages in which more drugs may enter the peripheral compartment resulting in large central volume of distribution.

The contribution of gender to the IIV of CL/F and the contribution of body weight and TPT levels to the IIV of V₂/F were marginal, reducing the IIV from 44.1% in the base model to 42.77% in the final model, and from 22.3% in the base model to 18.9% in the final model for CL/F and V₂/F, respectively. Therefore, none of these covariates (sex on CL/F, body weight and TPT on V₂/F) appears to be clinically relevant.

In conclusion, the population PK data described here suggested that systemic pomalidomide exposure is comparable between healthy subjects and patients with MM. However, MM patients may show deeper tissue/organ distribution of pomalidomide. Pomalidomide clearance is not complicated by demographics or baseline factors, other than gender. Furthermore, the finding that gender influences CL/F is unlikely to be clinically relevant, given that only 23% reduction in clearance was observed in female subjects.

### 7.2 Comparative Population Pharmacokinetic Analysis of Pomalidomide with Thalidomide and Lenalidomide

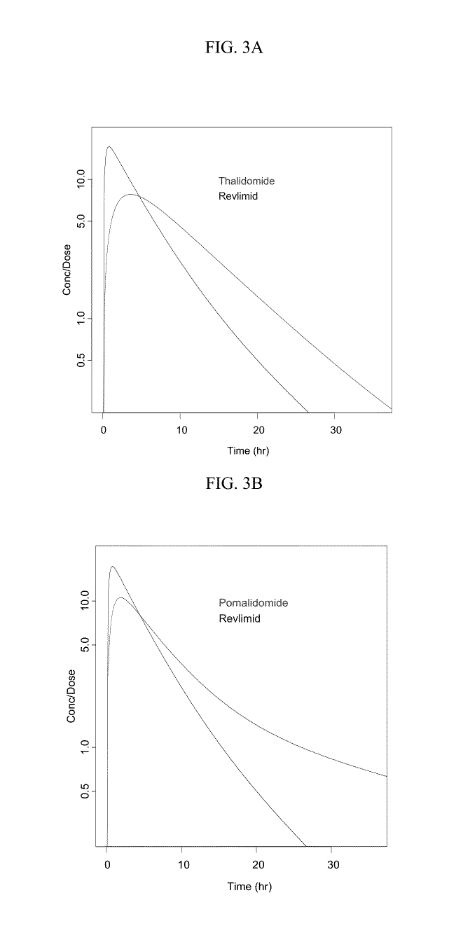

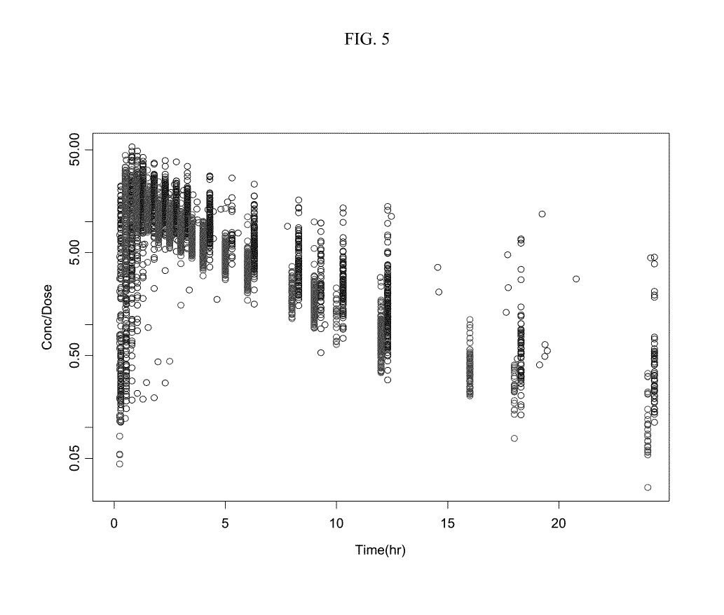

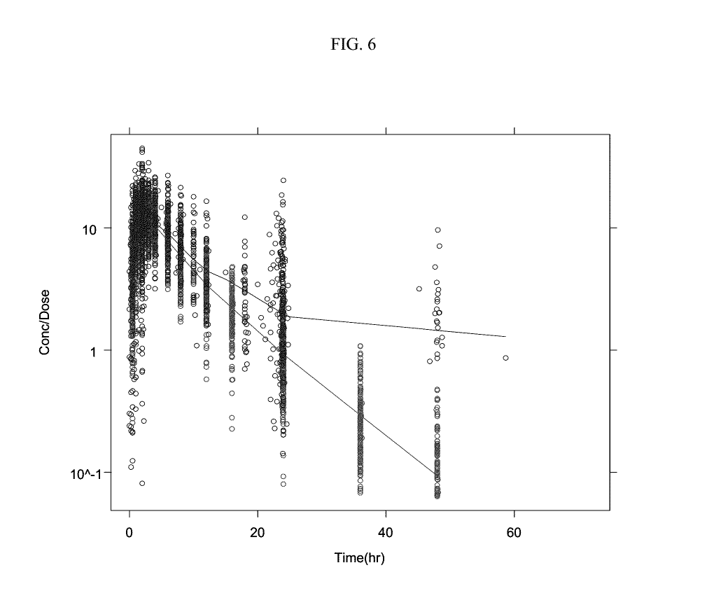

Population pharmacokinetic analysis of pomalidomide was compared with that of 2-(2,6-dioxopiperidin-3-yl)-1H-isoindole-1,3(2H)-dione (“thalidomide”) and 3-(4-amino-1-oxo-1,3-dihydro-2H-isoindo1-2-yl)piperidine-2,6-dione (“lenalidomide” also known as “Revlimid”), on available data from healthy subjects and MM patients. The results of analyses are provided in Table 7 and FIGS. 3A to FIGS. 9. FIGS. 3A and 3B provide concentration vs. time profiles for thalidomide, lenalidomide and pomalidomide.

The data of thalidomide and revlimid are based on a population pharmacokinetic analysis performed on data from studies using a two-compartment PPK model.

TABLE 7
Comparative PK in patients and healthy subjects

| | Thalidomide | Revlimid | | Pomalidomide | |
|---|---|---|---|---|---|
| | HNV | Patients | HNV | Patients | HNV |
| V₂/F (L) | 83.3 | 48.3 | 56.5 | 70.0 | 58.3 |
| V₃/F (L) | 13.5 | 8.36 | 9.49 | 71.49 | 8.45 |
| V3/V2 | 16% | 17.3% | 16.8% | 122% | 14.5% |
| Q/F (L/hr) | 0.355 | 1.13 | 2.14 | 3.75 | 1.01 |
| CL/F (L/hr) | 9.66 | 10.2 | 16.8 | 7.78 | 8.52 |
| Q/CL | 4% | 11.1% | 12.7% | 48% | 11.8% |
| KA (1/hr) | 0.536 | 4.57, 3 | 5.41 | 1.25 | 1.25 |
| T_{lag} (hr) | | 0.235 | 0.25 | 0.206 | 0.385 |
| Relative | 0.6% | 1.9% | 2.1% | 58.6% | 1.7% |
| Potential of | | | | | |
| Deep | | | | | |
| Penetration | | | | | |

PPK analyses results in Table 7 indicate that clearance and distribution kinetics of these compounds are comparable between healthy subjects and patients. A cross comparison of data for the tested compounds, clearly demonstrate the distinct tumor targeting properties of pomalidomide.

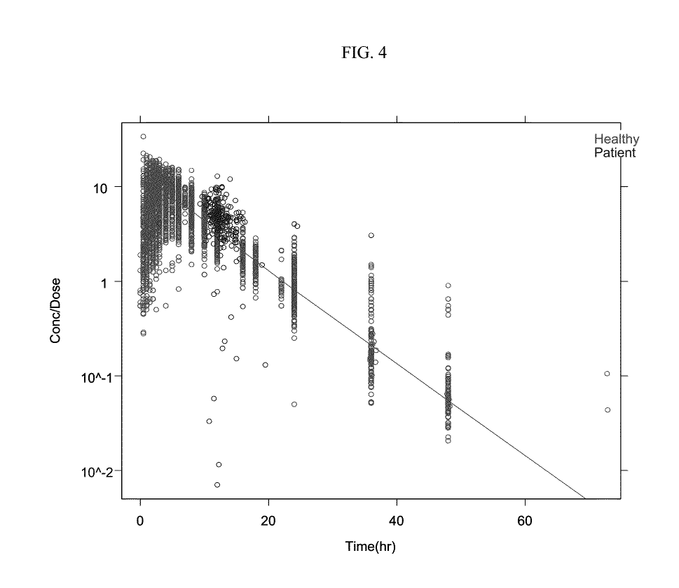

FIGS. 4, 5 and 6 provide concentration vs. time profiles for thalidomide, lenalidomide and pomalidomide, respectively in healthy subjects and MM patients.

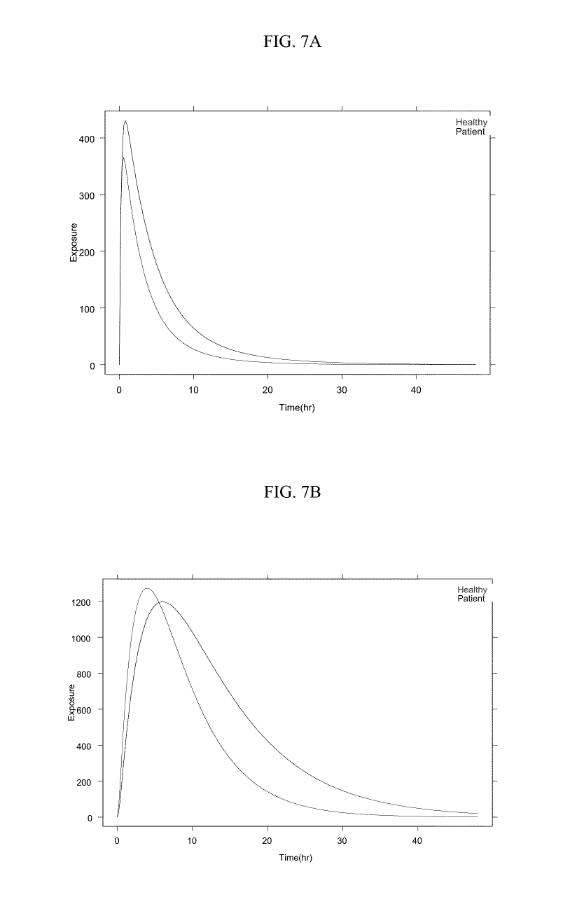

As shown in FIGS. 7A and 7B comparable drug exposures in both central and peripheral compartments in healthy subjects and patients was observed for lenalidomide.

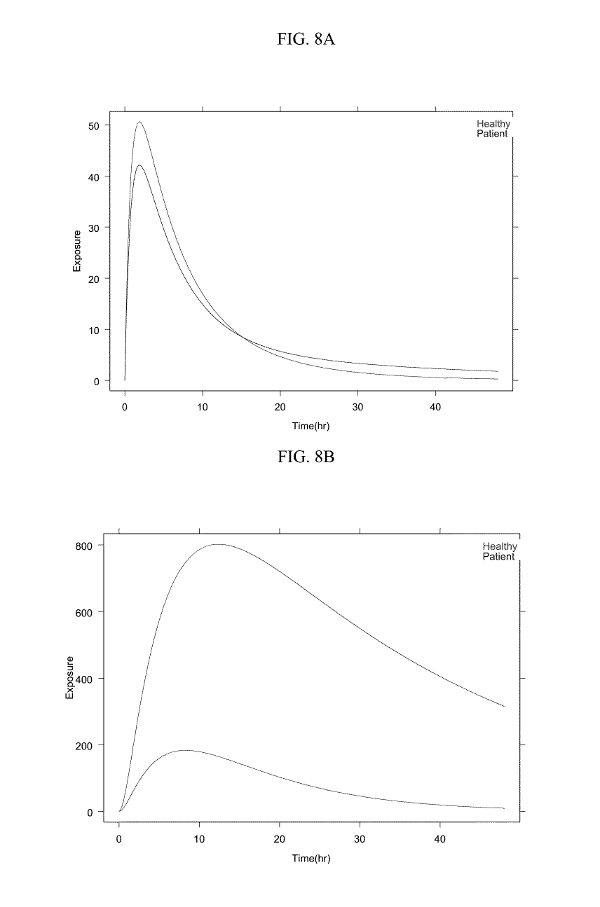

As shown in FIG. 8A, comparable drug exposures in central compartment was observed for patients and healthy subjects for pomalidomide. However higher drug exposure in peripheral compartments in patients as compared to healthy subjects was observed for pomalidomide as shown in FIG. 8B.

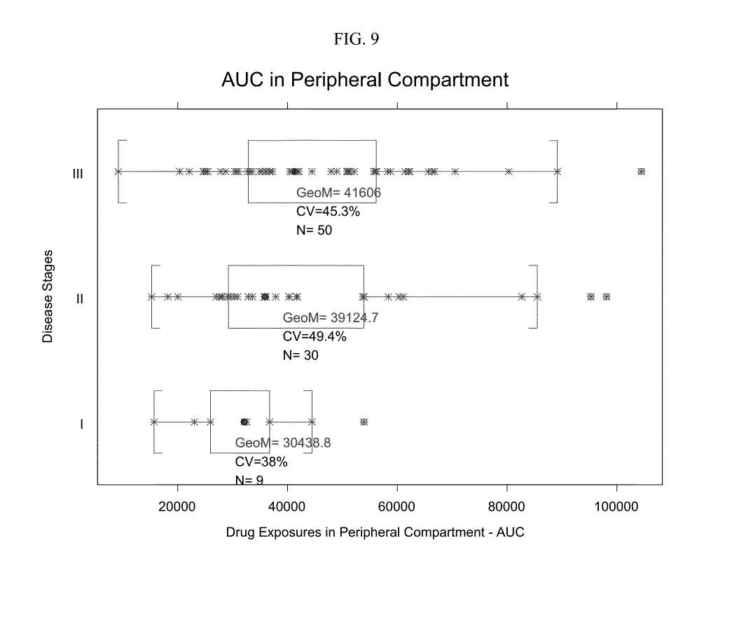

As illustrated in FIG. 9, clinical data for pomalidomide showed higher disease stage were correlated with higher peripheral compartment pomalidomide exposure in patients.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method of selectively targeting cancer cells, while leaving healthy cells intact, in a patient comprising administering to the patient in need thereof a therapeutically effective amount of a compound, wherein the compound is 4-amino-2-(2,6-dioxo-piperidine-3-yl)-isoindoline-1,3-dione, an enantiomer, a mixture of enantiomers, a pharmaceutically acceptable salt, solvate, hydrate, co-crystal, clathrate, or polymorph thereof.

2. A method of treating cancer while reducing an adverse effect associated with administration of a chemotherapeutic agent in a patient, comprising administering to the patient in need thereof a therapeutically effective amount of a compound, wherein the compound is 4-amino-2-(2,6-dioxo-piperidine-3-yl)-isoindoline-1,3-dione, an enantiomer, a mixture of enantiomers, a pharmaceutically acceptable salt, solvate, hydrate, co-crystal, clathrate, or polymorph thereof.

3. The method of claim 2, wherein the adverse effect is drowsiness, somnolence, dizziness, orthostatic hypotension, neutropenia, anemia, thrombocytopenia, fatigue, constipation, diarrhea, pneumonia, infections that result from neutropenia, increased HIV-viral load, bradycardia, Stevens-Johnson Syndrome, toxic epidermal necrolysis, and seizure.

4. The method of claim 2, wherein the adverse effect is neutropenia.

5. A method of enhancing selectivity against cancer cells as compared to healthy cells in a patient comprising administering to the patient in need thereof a therapeutically effective amount of a compound, wherein the compound is 4-amino-2-(2,6-dioxo-piperidine-3-yl)-isoindoline-1,3-dione, an enantiomer, a mixture of enantiomers, a pharmaceutically acceptable salt, solvate, hydrate, co-crystal, clathrate, or polymorph thereof, wherein selectivity is enhanced as compared to the selectivity obtained from a conventional chemotherapy.

6. The method of claim 5, wherein the conventional chemotherapy is a therapy with dexamethasone, lenalidomide, bortezomib or a combination thereof.

7. The method of claim 1, where the cancer is selected from bladder cancer, breast cancer, cervical cancer, colon cancer, esophageal cancer, head and neck cancer, liver cancer, lung cancer, melanoma, ovarian cancer, pancreatic cancer, prostate cancer, renal cancer, sarcoma, skin cancer, stomach cancer, testicular cancer, thyroid cancer, and uterine cancer.

8. The method of claim 1, wherein the cancer is relapsed, refractory or resistant to conventional therapy.

9. The method of claim 1, wherein rate of distribution of the compound to cancer cells is 2- to 10-fold higher as compared to the rate of distribution of the compound to healthy cells.

10. The method of claim 1, wherein rate of distribution of the compound to cancer cells is 4- to 8-fold higher as compared to the rate of distribution of the compound to healthy cells.

11. The method of claim 1, wherein the amount of the compound administered is from about 0.1 to about 50 mg per day.

12. The method of claim 11, wherein the amount of the compound administered is about 1, 2, 3 or 4 mg per day.

13. The method of claim 12, wherein the amount of the compound administered is about 4 mg per day.

14. The method of claim 1, wherein 4-amino-2-(2,6-dioxo-piperidine-3-yl)-isoindoline-1,3-dione administered is enantiomerically pure.

15. The method of claim 14, wherein 4-amino-2-(2,6-dioxo-piperidine-3-yl)-isoindoline-1,3-dione administered is S enantiomer.

16. The method of claim 14, wherein 4-amino-2-(2,6-dioxo-piperidine-3-yl)-isoindoline-1,3-dione administered is R enantiomer.

17. The method of claim 1, wherein the compound is administered orally.

18. The method of claim 17, wherein the compound is administered in the form of a capsule or tablet.

19. The method of claim 1, wherein the compound is administered for 21 days followed by seven days rest in a 28 day cycle.

20. The method of claim 19, wherein the compound is administered in an amount of about 25 mg per day for 21 days followed by seven days rest in a 28 day cycle.

21. The method of claim 1, further comprising administration of a therapeutically effective amount of a second active agent.

22. The method of claim 21, wherein the second active agent is antibody, hematopoietic growth factor, cytokine, anti-cancer agent, antibiotic, cox-2 inhibitor, immunomodulatory agent, immunosuppressive agent, corticosteroid, or a pharmacologically active mutant or derivative thereof.
